(19) United States
(12) Patent Application Publication
(10) Pub. No.: US 2007/0243137 A1
(43) Pub. Date: Oct. 18, 2007

(54) Cell and sub-cell methods for imaging and therapy

(75) Inventor: James F. Hainfeld, Shoreham, NY (US)

Correspondence Address: ABELMAN, FRAYNE & SCHWAB, 666 THIRD AVENUE, 10TH FLOOR, NEW YORK, NY, 10017, US

(73) Assignee: Nanoprobes, Inc.

(21) Appl. No.: 11/406,537

(22) Filed: Apr. 18, 2006

(51) Int. Cl. A61K 49/00; A61K 39/395

(52) U.S. Cl. 424/9.34; 424/184.1; 424/9.411

(57) ABSTRACT

Methods are disclosed to rapidly form and load cells and cell-derived vesicles. Loaded materials can include imaging agents, drugs and magnetic particles. Methods are also presented to additionally target the loaded cells or vesicles, leading to new forms of imaging, treatment, diagnosis, and detection by a large number of techniques. The preparation and use of reduced sized cells that retain subset characteristics of the parent cell are also described.

This application corresponds to Disclosure Document No. 570305, filed Jan. 14, 2005 and is incorporated herein.

### BACKGROUND OF THE INVENTION

1. Field of the Invention

This invention relates to a method for loading cells and cell-derived vesicles with contrast agents, drugs, or magnetic particles to enhance imaging or therapy. Also disclosed are methods to target the loaded cells or vesicles to specific sites using binding moieties or magnetic particles. The preparation and use of reduced sized cells that retain subset characteristics of the parent cell is also described.

2. Description of the Prior Art

Medical imaging is becoming an extremely important field since it can greatly aid in diagnoses and avoid more invasive methods such as exploratory surgery. A number of in vivo imaging devices have been developed based upon various principles, including X-ray, computed tomography (CT), fluoroscopy, magnetic resonance imaging (MRI), ultrasound, single photon emission computed tomography (SPECT), positron emission tomography (PET), infrared (IR) imaging, optical coherence tomography (OCT), florescent imaging, and confocal microscopy. These and other devices are constantly being improved to produce higher resolution, higher speed, and other desirable qualities.

Molecular markers, antibodies, peptides, drugs, nucleic acid probes, and other binding moieties have been widely used ex vivo to discriminate types of tissue abnormalities as well as detect types of bacteria and viruses. Many of these exquisitely sensitive tests require tissue material to be broken down (e.g., for DNA analysis), cut or permeablized to expose intracellular antigens, or be analyzed ex vivo due to limitations of the instrumentation, such as use of light, fluorescent, or electron microscopes, polymerase chain reaction (PCR) amplification, and other analytical requirements not compatible with in vivo imaging. Therefore, unfortunately, many of the molecular marker recognition techniques have not been successfully applied in vivo. Other complications arise in vivo, possibly including poor accessibility of the targets, confounding background biodistributions, toxicity of agents, lack of signal, and other problems.

Contrast Agents.

Contrast agents can enhance the imaging of certain tissues, compartments, or regions. Each imaging technique is generally associated with agents that give a unique or distinguishable signal. For example, X-ray and CT contrast agents are the iodine compounds, typically used during catheterization procedures of the heart and head; MRI agents are typically the gadolinium chelates; SPECT and PET agents are radioisotopes; and fluorescent microscopy uses fluorescent compounds or particles. Each of these has limitations when utilized in vivo.

Current X-Ray Contrast Agents

The currently available agents are mostly based on a benzene ring with 3 iodine molecules attached, with additional side chains for water solubilization. The first generation were ionic compounds, and pain was reduced by making them non-ionic, such as the popular iohexol (also called Omnipaque® or Exypaque®). High osmolality, which caused some of the problems, was reduced by making a dimeric compound, iodixanol (also called Acupaque® or Visipaque®). These agents are useful for coronary, cerebral, and renal angiography, but must be invasively administered arterially since their blood half life is very short. Data collection must be done immediately, and frequently the signal is nearly gone by the end of a CT scan.

Although iodine contrast agents have proven very useful, they have several drawbacks: 1) Imaging time is extremely limited. Iodine agents diffuse out of the vascular system rapidly and are therefore mostly used with invasive catheterization. 2) Non-invasive imaging from i.v. injection greatly reduces contrast from that obtainable from direct arterial administration, making this modality difficult, and 3) For non-invasive intravenously administered agent yielding low contrast, or repeated scans, for example in EKG-gated heart imaging, the X-ray dose to patient is elevated to improve signal, and may present a heath hazard and be tumorogenic.

Barium sulfate is successfully used to image the alimentary tract; but this cannot be injected intravascularly due to its toxicity (when in the blood) at the levels required for imaging.

Targeted X-Ray Agents

Another notable failure is that targeted delivery of X-ray contrast agents has not generally been successful since conjugation of iodine compounds to an antibody or peptide results in too few contrast atoms being delivered to the site of interest for imaging. Molecular targets on cells typically are expressed at less than 100,000 copies per cell. An iodine agent (carrying 3 iodine atoms) coupled to an antibody might optimistically achieve 5% binding, or 15,000 iodine atoms per cell. If one cell in 10 is an accessible target cell, this leads to an iodine concentration of ˜3×10e-8 M, which is too low to detect. Currently there are no FDA approved targeted contrast agents available for X-ray imaging and CT, even though they would be tremendously useful. Polymers have been explored to increase the number of iodine atoms per antibody, but these have been found to increase toxicity, and are bulky, limiting diffusion and access to many intended targets.

MRI Contrast Agents

The difference in native magnetic properties between different types of tissue is often insufficient to clearly distinguish the feature of interest in a magnetic resonance image. Lauterbur and co-workers were the first to demonstrate that paramagnetic substances may be used to change the magnetic properties of the tissue under study and improve the contrast between the feature under study and other tissues, and this led to the application of paramagnetic and superparamagnetic substances as contrast agents.

Contrast agents change the relaxation times of nearby hydrogen atoms, thus enhancing or attenuating the signals from different types of tissue. The criterion for an effective contrast agent is a large magnetic moment, and this is met by gadolinium (Gd), a highly paramagnetic lanthanide with seven unpaired electrons. The most widely used contrast agents are chelates of trivalent Gd. In the presence of gadolinium ions (Gd³⁺), relaxation times of ¹H are shortened dramatically, resulting in large differences in image intensity between tissues containing gadolinium and those without. However, Gd³⁺ is toxic when injected at a concentration sufficient for MRI imaging. Toxicity is reduced by chelation, and the first intravenous contrast agent approved for human use was gadolinium diethytriaminepentaacetic acid (Gd-DTPA), which was used for brain and spinal imaging. The ionic properties of this compound, however, are not ideal for all applications. It does not cross the blood-brain barrier, and is rapidly excreted by glomerular filtration. Furthermore, some side-effects have been attributed to its hyperosmolar properties. More recently, non-ionic gadolinium agents have become available. Gadolinium diethylenetriaminepentaacetic acid bismethyl-amide, Gadodiamide (Omniscan, Nycomed-Amersham), introduced in 1992 as the first non-ionic MRI product, and Gadoteridol (ProHance, Bracco) are examples of such compounds. These exhibit lower toxicity and lower incidences of side-effects than the ionic chelates. Non-ionic chelates have become the reagents of choice for brain imaging.

However, these are not ideal for all applications. Since they are small molecules, they are relatively quickly removed from the vascular system In addition, a large number of lanthanide atoms are required to generate sufficient signal for effective imaging (10 to 100 μM). Only a very small number of chelates may be conjugated to an antibody without compromising immunoreactivity: therefore, targeted lanthanide reagents with sufficient lanthanide loading to selectively image a feature of interest, such as a tumor, are not feasible. Use of polymers and larger vehicles has generally increased toxicity, or increased clearance by the liver and reticuloendothelial system, thus again preventing achievement of targeted imaging. Larger superparamagnetic iron oxide nanoparticles have been used as contrast agents for gastrointestinal imaging; these are retained longer and have a significantly greater effect, but lack of a reliable conjugation chemistry, the size of the nanoparticles hindering binding to its target, and their higher toxicity have clinically restricted their use to gastrointestinal imaging.

Because MRI is non-invasive, a number of important applications loom on the horizon for its expanded use. Instruments are always improving, giving better resolution and sensitivity. Some of the desirable applications that perhaps could be achieved with MRI and better contrast agents include:

1. Molecular imaging: If antibodies, peptides, or other targeting molecules could deliver enough of a contrast agent to specific tissues, many more conditions could be usefully imaged using MRI. Of the many examples: Antibodies to tumors could detect smaller tumors and better localize tumors for image guided procedures or tracking therapies, staging of tumors and identification of types that could respond to specialized therapy (e.g., Herceptin, a drug to treat breast cancers that overexpress Her-2/neu protein). Vascular plaques could be identified and antibody to fibrin or p-selectin could better image blood clots in stroke, and in peripheral clots before they become pulmonary embolisms or create strokes.

2. Multicolor MRI: Two or more molecular targets could be distinguished if probed for at the same time. Much like fluorescence, it would be desirable to have “multicolor” MRI contrast agents.

3. Angiography: Currently X-rays dominate this field, but catheterization and exposure to X-rays make this procedure invasive and expensive. A significant advance would occur if MRI using intravenously administered agents could achieve comparable data non-invasively (Magnetic Resonance Angiography, MRA).

4. Intraoperative MRI: During surgery, real time imaging can assist the surgeon to visualize tissues of interest. MRI machines that enclose an operating theater are currently being produced. Better contrast agents could greatly aid in this setting.

Blood Pool Agents

MRI is a good non-invasive imaging method, but the standard Gd-DTPA and Gadodiamide clear the vascular system rapidly through rapid kidney clearance and leakage across the endothelial barrier in most organs with a blood half-life of ˜20 min and are not ideally suited as blood pool agents. An agent that had a longer blood half life and no toxicity or unwanted biodistribution accumulation would be valuable in assessing coronary arteries, stroke, carotid arteries, atherosclerotic plaque and stenoses, renal function and other vascular defects and conditions. Direct imaging of in these cases now requires invasive catheterization. It is desirable to achieve non-invasive reliable detection of perfusion defects on first pass and equilibrium perfusion imaging and characterization of viability after myocardial infarction or stroke and to perform a comprehensive cardiac/cerebral MR examination. Although several experimental blood pool agents have been evaluated including gadolinium bound to proteins or polymers, and iron particles, and much effort expended to achieve this goal, no blood pool MRI agent has been FDA approved.

Vesicle and Cell Encapsulated Materials

Previous work has been done demonstrating that various agents useful for contrast or other uses can be produced by encapsulating the materials in synthetic or cell derived vesicles. These may provide extension of blood half-life for extended imaging times, for example. For MRI contrast agent applications, liposomes were used to encapsulate gadopentetate dimeglumine (Bednarski et al., Radiology. 1997;204(1):263-8). Oligodendroglial progenitors were loaded with iron particles by receptor mediated endocytosis and tracked in vivo by MRI (Bulte et al., Cereb Blood Flow Metab. 2002;22(8):899-907). Transfection agents were incubated with ferumoxides and MION-46L in cell culture medium to get iron particles into cells (Frank et al., Radiology. 2003;228(2):480-7). In 1998, loading of intact-sized red cells by osmotic pulse in the presence of gadolinium DTPA dimeglumine was reported to produce a blood pool agent (Johnson et al., Magn Reson Med. 1998; 40(1):133-42). This group also loaded red cells with dysprosium DTPA-bis-methylamide (Johnson et al., Magnetic Resonance in Medicine 45:920-923, 2001). In summary, synthetic liposomes encapsulating magnetic materials have been tried, as well as cells loaded with iron particles internalized by endocytosis or transfection agents. Red cells were also loaded with paramagnetic compounds. For X-ray absorption, loading of red blood cells with metal particles had been described (Hainfeld, U.S. Pat. Nos. 6,645,464, 5,690,903, and 5,443,813).

Heart Disease

There are 1.1 million heart attacks each year resulting more than 500,000 deaths in the U.S. alone (it is the number 1 killer). A non-toxic contrast agent could greatly reduce this number by detecting problems while still treatable. Heart attacks typically occur after a coronary artery is narrowed by years of plaque deposit, which suddenly ruptures, initiating a blood clot. There is about 10 minutes to get help, longer than an ambulance response. Many people are currently at high risk, but do not know it. Although cholesterol and stress tests are of some use, coronary angiography remains the standard for assessment of anatomic coronary disease, because no other currently available test can accurately define the extent of coronary luminal obstruction. Because the iodine dyes only show arteries for a few seconds before they diffuse out of the vasculature, this procedure requires snaking a catheter through a leg artery to the heart for injection of the dye, with X-ray dose to visualize it. Unfortunately, this can result in puncture of an artery, dislodging plaque causing a heart attack or stroke, or anaphylactic shock from the dye. Statistically, one in 600 die of the procedure alone, and one in 59 have major complications. It is also expensive, the procedure costing about $6,000, and requiring highly trained physicians. A non-toxic contrast agent that remained in the vasculature long enough for imaging in the heart would greatly aid in assessing the condition of the coronary arteries, since it could be injected intravenously by a nurse in the arm, for example, without risk, and at a far lower cost.

It is estimated that greater than 15 million people in the U.S. are at serious risk of an impending heart attack, but are completely unaware of their life-threatening condition. Use of an effective, non-invasive, and economical contrast agent would permit advance identification of persons at risk. Subsequent treatment by diet, exercise, drugs, or surgery could then prevent many fatal heart attacks.

Stroke

Stroke is the third leading cause of death in the Western world and is the most common cause of neurological disability. It is important to develop tools to study, prevent, treat, and monitor treatment of this condition. Many strokes are caused by atherosclerosis in the carotid arteries that at some sudden point become occluded or send fragments that occlude smaller brain vessels. Current assessment of plaque and stenosis is done by invasive and expensive angiography. A non-invasive procedure using MRI, or MRA “magnetic resonance angiography” has long been sought, where a simple intravenous injection of agent is administered followed by MRI. The condition of the carotid arteries and other brain vasculature could then be clearly visualized by MRI.

The sought after agents could improve delineation of cerebral vascular malformations, for example arterio-venous anastamoses and aneurisms. Detailed visualization of stroke circulation and reperfusion and hemorrhaging would be possible with good spatial resolution to better treat strokes in progress. Atherosclerosis could be seen by visualizing stenoses.

It would also be desirable to achieve molecular targeting, where vulnerable plaque could be distinguished from stable plaque, and enable the physician to decide what form of treatment is needed to prevent stroke (or myocardial infarction).

Tumor and Vulnerable Plaque Vascularity

Tumor vascularity is highly predictive of tumor aggressiveness and prognosis. Core biopsies (which are invasive) are just samples, and do not accurately reflect the overall tumor, limiting their potential as predictive or prognostic markers. A non-invasive imaging technique which visualizes tumor vascularity in vivo would overcome these limitations.

Vulnerable plaque has a higher degree of vascularization, and it would be desirable to have a non-invasive method to quantify the vascularization of plaques. This could be done with the agents disclosed herein.

Lymphography—Detection of Sentinel Lymph Nodes

High resolution contrast enhanced lymphography after interstitial or intravenous injection would be another major step forward in diagnostic imaging. The sentinel lymph node is the first lymph node to receive drainage from a tumor site. Analysis of this node is highly correlated with the spread of the disease, prognosis, and treatment prescribed. Radioscintigraphy, PET, blue dye, and surgical resection and histology are used, but would be improved by non-invasive MRI or CT. In Europe, radiolabeled nanocolloids are injected, but in the U.S. sulfur colloid is preferred. In breast cancer the agents are injected peritumorally or periareolarly, and flow into the sentinel lymph node. The particles or colloids used range in size from <0.22 to 2 microns. Several benefits would accrue over current lymph node imaging if appropriate agents, such as disclosed herein, were available: a) the location would be precisely determined for surgery or biopsy since MRI and CT are high resolution compared to SPECT or PET now used; b) no radioactivity is needed; c) deep lymph nodes would be visible, a problem now with the blue dye technique; d) their enlargement would indicate extent of tumor metastases; e) antibody-conjugated contrast agents could be prepared to molecularly image the tumor for ascertaining positive lymph node involvement and discern the tumor type for selecting the best treatment; f) with such a simple technique that exquisitely images the sentinel lymph nodes, better diagnoses, image guided interventions, treatments, and therapy monitoring would be realized for many cancers.

Therapy and Drug Delivery

Many therapeutic substances are known that can kill bacteria, kill tumor cells, or that could potentially alleviate symptoms, and favorably alter the course of a disease or condition. Unfortunately, these substances frequently affect and harm normal tissues, leading to a severe toxicity before the intended effect is achieved. For example, there are many cytotoxic drugs that can kill tumor cells. Administration, however, can cause gastrointestinal problems, damage to the immune system, neurological problems, and other severe side effects and sickness, such that a dose cannot be given that will eradicate the cancer. Radiation has enough power to kill tumor cells. Here again, normal tissues are also affected, and most commonly, a radiation dose that will completely kill the tumor would also kill the patient. Therefore, a lower, somewhat effective palliative dose is given, that may prolong life for a limited period. Radiation effects are cumulative, thus limiting the total dose that can be given, frequently ruling out needed retreatments.

Much of the difficulty with drugs is that they are not confined to the region of disease, thus imposing their toxic effects on sensitive normal tissues. Drugs administered intravenously, orally, or intraparitoneally typically disseminate throughout the body and experience not only dilution but uptake in various tissues. Effectiveness of local injection or administration of drugs to a target site is beleaguered by entry into the blood or lymphatics thus spreading the drug, and mistargeting to surrounding or interspersed normal tissue. In many cases of disease or maladies, it is not the lack of drugs or methods to kill or alter cells to achieve effectively treatment, but the lack of specific delivery to only the target cells. Drug delivery is perhaps the single most limiting factor in treatment of diseases.

Drug targeting has been accomplished to varying degrees of success using a variety of techniques. If a drug is reasonably specific for the target, its effects will be so localized. Antibodies, peptides, aptamers, and any other substances that bind reasonably specifically to target cells have been attached to drugs for selective delivery. Magnets have also been used to attract magnetic particles associated with drugs. Direct injections and other local applications are sometimes employed to localize drugs.

Natural body cells, such as NK killer cells, CD8+ lymphocytes, macrophages, and other cells are involved with the normal body's defense against infections and diseases. Certain methods have been developed to mobilize these defenses, such as the administration of cytokines or challenges with BCC virus to heighten the immune system. Adoptive immunotherapy extracts particular lymphocytes that can affect tumors, proliferates these cells ex vivo, and then reinjects them to the patient to provide a large number of specialized cells. Although sometimes effective, this method still is plagued by many barriers such as tumor localization, crossing the vascular barrier, and low immunoreactivity of the tumor.

Vesicles and Cell Loading for Imaging and Therapy

Some of the obstacles in imaging and therapy might be overcome by packaging the contrast agent or therapeutic drug in a vesicle or cell so that more is delivered to the site of interest. This has the advantage of a “payload” of material being carried rather than use of single small molecules. A number of previous reports describe various systems along this line, but all continue to have shortcomings as evidenced by the absence of FDA approved clinical products, long after these “promising” ideas were disclosed. Closer examination of these approaches reveals a number of drawbacks.

WO 85/00751 discloses the loading of drugs into liposomes and that these liposomes can be targeted by attaching antibodies to their surface. The use of liposomes imposes a number of disadvantages: a) liposomes are not normal physiological substances and are subject to immunological rejection by the patient; b) liposomes have short blood half lives since they are recognized by the reticuloendothelial system in the spleen and liver and rapidly removed; even though longer lasting liposomes (called “stealth liposomes”) have been developed, the blood half life then generally is extended from a few minutes to several hours. This is still very short compared to erythrocytes that last 120 days. c) Liposomes have no water channels, thus substantially reducing signals of MRI T₁ contrast agents. d) Liposomes bear some toxicity, limiting their use.

Gamble et al. (U.S. Pat. No. 4,728,575) discloses micellar vesicles that can have antibodies attached to encapsulate and deliver MRI contrast agents. Significant problems with the micellular particles of Gamble et al. include: a) they cannot enclose large amounts of paramagnetic materials, b) they are subject to immunological rejection, c) they are devoid of water channels, reducing signal, d) they remain in the blood for very short times due to their excretion and efficient removal by the reticuloendothelial system, e) micelle particles bear some toxicity, limiting their use.

Unger et al (U.S. Pat. No. 5,542,935) describe microspheres in connection with imaging, therapy, and application of external energy. The basic idea behind the Unger et al. patent is to make liposomes containing a liquid and contrast or therapeutic substance, which when exposed to preferably ultrasonic (or other forms of) energy; the liquid will heat up and become a gas, thus rupturing the vesicle and release the contrast or therapeutic substance (Unger et al, Abstract, col 4, lines 26-56). Several severe restrictions of this method are that synthetic liposomes are required and a precursor gas material must be included in the liposome such that it is administered below its phase transition, then upon heating above its phase transition it becomes a gas. This is difficult to practically control. Unger et al. teach loading of gas-liposomes with metal ions, but not with metal particles. This can severely and adversely limit loading and stability. Unger et al. do not disclose the use of X-rays, gamma rays, or proton beams for therapy since their gas-liposomes do not contain metal particles appropriate for secondary production. The gaseous microspheres of Unger et al. bear some toxicity, limiting their use.

Filler et al. (U.S. Pat. No. 5,948,384) disclose methods to image or deliver drugs to nerves, but their methods require that the agent (which could be a liposome) be specifically targeted to and taken up by living nerves and additionally, their agent must be capable of axonal transport. They accomplish this by combining a nerve adhesion molecule (NAM), which is required, with a physiologically active or diagnostic marker, but the latter must be capable of axonal transport. These restrictions severely limit more general diagnostic imaging and drug delivery. The liposome-drugs described by Filler et al. bear some toxicity, limiting their use.

Watson et al. (U.S. Pat. No. 5,688,486) describe the use of Fullerenes and Fullerene-like branched carbon mesh capsule structures as carriers for diagnostic or therapeutic agents, including diagnostic contrast agents. Agents would be attached to the Fullerene-like structure either by covalent attachment, substitution for atoms in the framework, intercalation between adjacent webs, or entrapment in a Fullerene cage. Release of agent is also described if it is held loosely or can diffuse out of the Fullerene structure. The Fullerene-like carrier structure is absolutely essential for all applications and uses disclosed by Watson et al. However, the Fullerenes have severe limitations, such as the amount of agent that can be carried. For example, the number of metal atoms carried is listed in claim 6 to be 1, 2, 3, or 4, or more limiting, in claim 6, only 1 or 2 per Fullerene. This is also born out in the examples given. Although this may be of utility for radioactive imaging where low concentrations are acceptable, this approach will not be suitable to deliver the much higher concentrations of agents needed for MRI or X-ray targeted imaging. Fullerenes and their conjugates described by Watson et al. bear some toxicity, limiting their use. Hainfeld (U.S. Pat. Nos. 5,443,8138 and 5,690,903) discloses loading of molecules, viruses, and cells with and without targeting moieties attached for the purposes of diagnosis and therapy. With respect to cells, this patent specifically restricts itself to full-sized, naturally occurring cells or full-sized membranes from cells that have been depleted of their normal contents. Such full-sized cells will not penetrate well into tumors, kidneys, lymph nodes, and other regions of interest that are outside the vascular system. Therefore, the imaging and delivery of therapeutic materials to such important targets will be severely limited. The main focus of these patents is loading uranium into the protein apoferritin, which is not an aspect of this application. Although loading of cells is discussed, loading by freeze-thawing and vesicle or cell fusion are not taught, nor is growth by vesicle or cell fusion.

Hainfeld (U.S. Pat. No. 6,645,464) describes loading seed metal nanoparticles into red blood cell (erythrocyte) vesicles, then growing these seeds by catalytic metal deposition, then using the vesicles for imaging or therapy. This disclosure requires that metal seed particles be introduced into vesicles and necessitates a chemical process to deposit additional metal on the seed particles. This has several disadvantages: a) only certain seed metal nanoparticles and specific deposition metals will work with this method; b) only metal in the zero oxidation state is produced, which is not generally suitable for MRI contrasting; c) there are multiple steps involved in forming the product thus complicating synthesis.

Johnson et al. (Magn Reson Med. 1998, 40:133-42) described loading of whole red blood cells with a gadolinium salt for use as a blood pool MRI contrast agent. Further work by Johnson et al. then demonstrated loading of whole red blood cells with a dysprosium salt, also for MRI contrasting (Magnetic Resonance in Medicine 45:920-923, 2001). Their methods used hypotonic lysis which necessarily limits the loading of the cells to a low value. They achieved 28-30 mM Gd or Dy inside the cells. It would be desirable to have a higher concentration of contrast agent incorporated, but this was the maximum that they found possible with their methods. Several drawbacks of this effort were: a) low incorporation of contrast agent, b) no targeting was demonstrated or described to guide the loaded blood cells to a specific site; c) only full sized red blood cells were loaded, thus severely limiting their access to tumor cells, lymph nodes, and other tissues due to their large size.

### SUMMARY OF THE INVENTION

This invention discloses methods to load cells and cell-derived vesicles with contrast agents, drugs, magnetic particles, or other substances to enhance imaging or therapy. Targeting of the loaded cells or vesicles to sites of interest by attachment of surface binding moieties and use of magnetic fields is also disclosed. The preparation and use of reduced sized cells that retain a subset of characteristics of the parent cell is also described.

### BRIEF DESCRIPTION OF THE DRAWINGS

The drawings show:

FIGS. 1-13 form part of this application and accompany it with explanatory text.

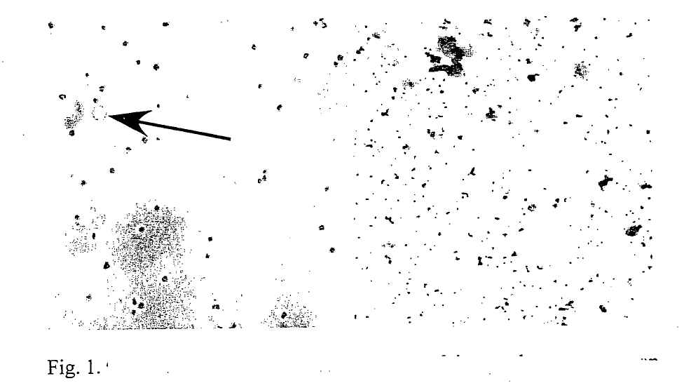

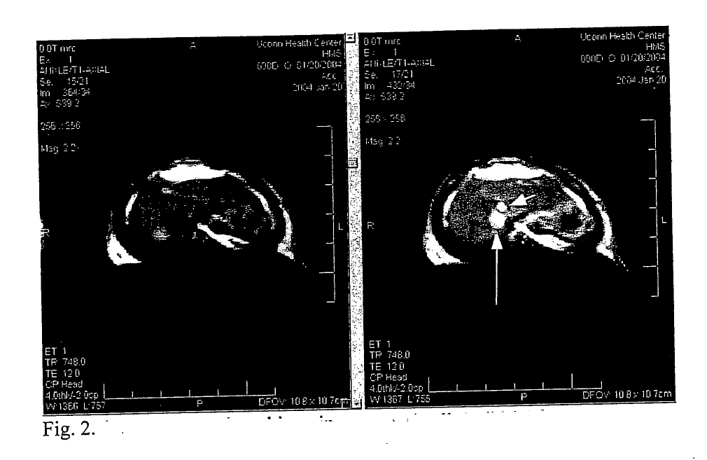

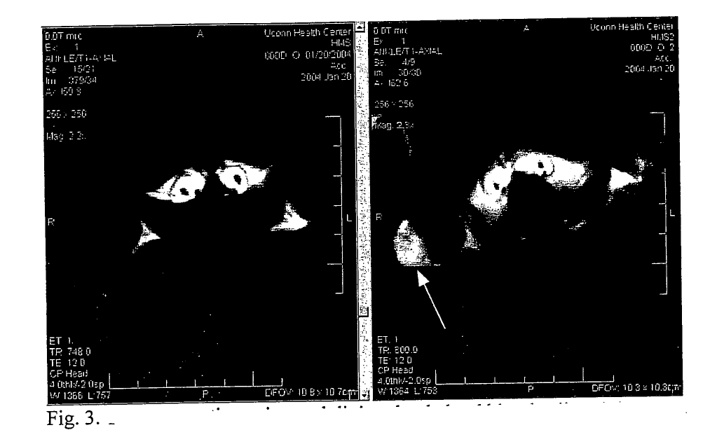

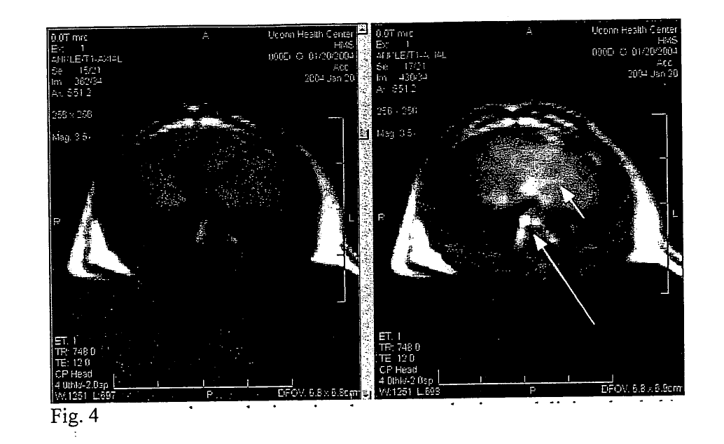

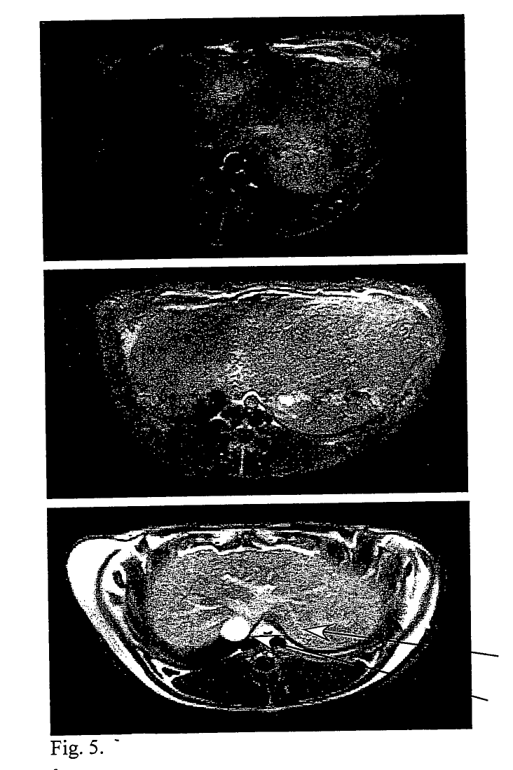

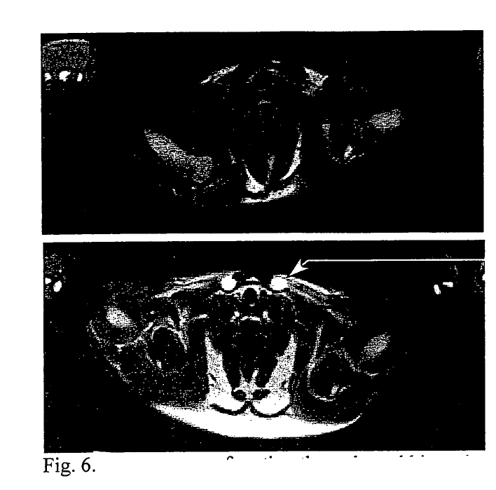

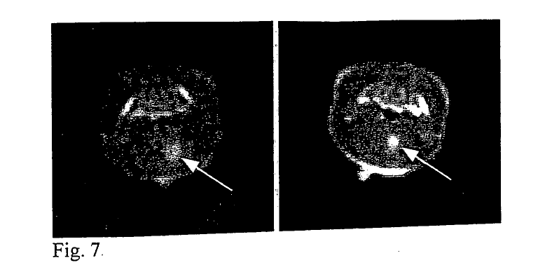

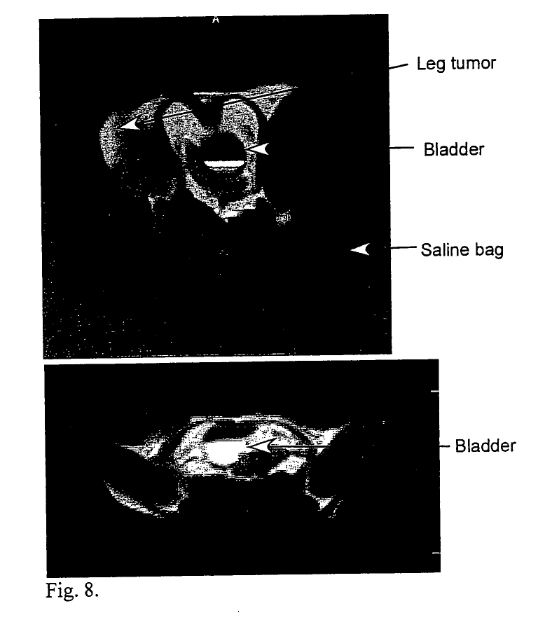

### DEFINITIONS

“Vesicles” as used herein refers to lipid bilayer or multilayer1 bounded volumes. This includes synthetic vesicles, frequently termed “liposomes” as well as cells and smaller or larger constructs that include cellular membranes or membrane components.

### DETAILED DESCRIPTION OF THE PREFERRED EMBODIMENTS

Synthetic lipid vesicles have been used to encapsulate drugs. While most liposomes have a half-life in the blood of only minutes, liposomes with a more biocompatible choice of phospholipids can prolong the blood half-life to about one day. In contrast, red blood cells remain in the blood for 120 days and may function as improved drug carriers. Here we describe a method to use natural cells and cell-derived vesicles to encapsulate desired cargo. Although advantages are obtained by using natural cells, in some instances synthetic vesicles may be preferable, and these too may be prepared by the methods disclosed.

Contrast Agents

In the past 25 years few contrast agents have been FDA approved for use. One reason is that many injectates are toxic or do not clear the body well at the amounts needed for good imaging. The present invention overcomes many of the obstacles of other approaches and materials and provides novel contrasting for enhanced medical imaging.

Red Blood Cells

Red cells are plentiful and can be obtained from blood banks or a patient. However, a convenient method to highly load them with materials has not been developed. Here we show how blood cells can be easily and conveniently loaded. This then facilitates the clinical usage of such methods to extract cells from a patient by venipuncture, quickly process them for loading, followed by reinjection for imaging or therapy, all within a short period of time. Although other blood can also be used, use of a patient's own blood avoids the risks of disease transmission such as HIV-AIDS, hepatitis, and other blood borne pathogens.

Whole blood is preferably washed by low speed centrifugation to obtain the cell fraction in the pellet and isolate cells from serum proteins. Simple sedimentation, dialysis, column chromatography, or other methods may also be used. A physiologic buffer, such as PBS (phosphate buffered saline) or saline, can optionally be used to resuspend the cells or wash them additional times. It is preferable to concentrate the cells to be loaded to maximize loading, but this is not absolutely required. The cells are then mixed with the material to be loaded. It is convenient to have the material to be loaded in concentrated form to maximize loading. Unless lysis is desired at this point, the final ionic strength must be within a range to prevent lysis when reinjected into the patient. This can be conveniently adjusted with salt or other substances. Actual loading of the cells is accomplished by a variety of techniques, including hypotonic lysis, electroporation, sonication, detergent treatment, receptor mediated endocytosis, use of protein transduction domains, particle firing, membrane fusion, freeze-thawing, mechanical disruption, and filtration. For hypotonic lysis, the cells are exposed to a low ionic strength environment causing them to burst. The loading material then distributes within the cell, and the cell (or ghosts) can be resealed by addition of salt and/or gentle heating. For electroporation, electric impulses are applied which cause transient holes in the cell membrane, thus allowing the material to enter. For sonication, cells are subjected to high intensity sound waves, causing transient disruption of their membranes, during which the material can enter. For detergent treatment, an appropriate detergent is applied which transiently compromises the cell membrane or creates transient holes in it. After loading, the detergent is removed (for example by centrifuging the cells). For receptor mediated endocytosis, the material to be loaded contains a moiety that binds to a cell surface receptor. The receptor and its contents may then be internalized. A protein transduction domain (PTD) (for example, the TAT peptide sequence from the AIDS virus, the Drosophila Antennapedia (Antp) homeotic transcription factor sequence, and the herpes-simplex-virus-1 DNA-binding protein VP22 sequence) may be attached to the material to be loaded and the PTD enhances intracellular delivery. For mechanical firing, the substance to be loaded may be optionally attached to heavy or charged particles which are mechanically or electrically accelerated such that they traverse through the target cell membranes, which then reseal. For membrane fusion, the material to be loaded is contained or associated with synthetic vesicles, which under conditions that enhance vesicle fusion cause fusion with the cell membrane and loading of the material. For filtration, the cells and material are passed through pore sizes smaller than the cell, causing transient membrane disruption, permitting loading. For freeze thawing, the cells are frozen, then thawed one or more times, resulting in cell disruption, especially by ice crystal formation damage. For mechanical disruption, the cells are agitated powerfully enough against hard surfaces to cause membrane breaches.

A preferred technique is the use of filtration because of its simplicity and surprising effectiveness. If pore sizes are chosen that are somewhat smaller than the cells, vesicles from the cell membranes form that are consistent with the pore size, i.e., small pore filters create small vesicles. The ultimate size of the vesicles can be controlled by the filter pore size, and more uniform vesicle size can be obtained by multiple passes through the filter. Vesicles were found to be highly loaded and therefore must go through a stage where the membrane is breached before it reseals, allowing influx of the material to be loaded. Vesicle size affects blood half life, tumor uptake, leakage through tumor or angiogenic vasculature, pharmacokinetic biodistribution in the kidney, liver, lung, and other organs and tissues. This filtration technique provides a method for easily controlling many pharmacokinetic properties.

Some of the other methods referenced above can also produce vesicles of varying size, e.g., sonication and detergent treatment.

The loaded cells or vesicles may then be used directly, by, for example intravenous injection into a patient, or purified further to remove unincorporated material or other substances. This may be accomplished by differential centrifugation, dialysis, sedimentation, column chromatography, electrophoresis, or other means. However, if the material to be loaded is not toxic (at the concentrations used), it may be acceptable or preferable to skip this purification and inject the loaded cells or vesicles with free, unincorporated material. There will be a further time and tissue separation of the two phases in vivo as the body separates and removes the two at different rates, but this may not interfere with the intended goal, and may in fact provide a dual phase cocktail that has the advantages of both free and encapsulated material.

A preferable method is to collect a sample of blood from a patient, gently pellet the red cells, remove the supernatant, mix with concentrated material to be loaded, pass through a filter with pore size less than 8 microns, and reinject the filtrate.

Another preferable method is to utilize the surprising discovery that freezing the cells, then thawing them leads to formation of smaller vesicles, which if the material to be loaded is present during this process, it becomes encapsulated while the cells are broken and reforming into vesicles. Freezing and thawing at various rates affects the final vesicle size and time that the membranes are breached. A time may be chosen for this process to permit the desired amount of material to be loaded to be encapsulated in the final vesicles. Freezing rates can be controlled by a number of means including rapid freezing with liquid ethane or propane, liquid nitrogen, dry ice-acetone, dry ice-isopropanol, or slower cooling by refrigeration at various low temperatures, and even thermocouple controlled cooling for precise rates. Thawing can be controlled by a number of means including immersion in warm water, warming in air, or more controlled environmentally-controlled warming such as temperature controlled baths that increase the temperature at a known rate. The number of cycles of freeze-thawing can affect the final size of the vesicles and the efficiency of incorporation of the loaded material. It was found that red cells mixed with isotonic agent to be loaded then frozen in liquid nitrogen followed by thawing in 23° C. water bath for three cycles led to well-loaded 0.1-0.2 μm vesicles. A preferable method is then to collect a sample of blood from a patient, gently pellet the red cells, remove the supernatant, mix with concentrated material to be loaded, freeze and thaw, optionally multiple times, and reinject the product. The vesicle product can be optionally isolated by dialysis, filtration, centrifugation, or other means if desired.

Depending on the age of the blood, one freeze-thaw cycle in liquid nitrogen can result in moderate but not complete permeation of the cells, with little change in their size. A second treatment can result in most of the cells becoming permeable without significant size change. Multiple cycles typically increase the percentage permeablized (and therefore loaded), but with more smaller than original cell sizes produced. Fresh, washed blood typically is nearly completely loaded with the surrounding medium material with two liquid nitrogen freeze thaw cycles while maintaining a significant number of vesicles greater than 1 micron.

Resealing of the vesicles after some permeation method is important so that the loaded material does not escape. Sealing can be enhanced by treatment at about 150 mM salt, pH 5.5 and with increasing temperature and time. Treatment at 60 degrees C. for 1-2 minutes under such conditions generally results in well-sealed membranes. However, sealing at different temperatures (20-100 degrees C.) and other salt and pH conditions may be used. Higher temperatures and times may result in additional aggregation, membrane fusion and possible denaturation, so must be carefully used.

Although cell loading by various means has been previously described, this new method provides a significant enhancement in speed, concentration of loaded material achieved, and clinical feasibility.

Mechanical disruption was surprisingly found to produce highly loaded vesicles from red blood cells. Erythrocytes may be loaded into a container with the solution or suspension to be loaded and also with stainless steel balls or other hard objects. A mechanical shaker or other such device may then be used to produce mechanical stress strong enough to break the cells, thus allowing the material to be loaded to enter the open membranes. When the membranes reform into vesicles, they now contain the substance to be loaded. Other forms of mechanical disruption include, but are not limited to: passage through a small bore needle or tube and compression between surfaces, such as optical flats or glass, metal, or plastic plates.

A method to efficiently load erythrocyte membranes or other cell membranes has been found. Cells are first washed in isotonic buffer, for example, 5 mM sodium phoshphate buffer pH 8 with 150 mM sodium chloride. This may be accomplished by centrifuging the cells and discarding the supernatant, along with the “buffy coat”, or top layer of the pellet that contains other cells. This operation is preferably done two or three times. The cells are then hypotonically lysed by adding an approximate 40 fold volume excess of low ionic strength, for example, ice cold 5 mM phosphate buffer, pH 8. Cell membranes are then isolated in concentrated form, for example by centrifugation. The supernatant is discarded as well as the hard part of the pellet that contains other cell types and unlysed cells. This operation is preferably done only once. The material to be loaded is then added in concentrated form, preferably also in a low ionic strength solution, to the purified membranes and incubated with them, preferably on ice, preferably for 30 minutes, although other times from 1 min to several days may be used. The mixture is then adjusted to approximately 150 mM in salt, for example by adding a concentrated buffer such as 100 mM phosphate, pH8, containing 3 M sodium chloride, so that the final concentration is 150 mM sodium chloride. The mixture is then incubated at a warm temperature, from 25-50 degrees C., preferably at about 37 degrees C. for 5 minutes to 4 hours, preferably for about 30 minutes. The latter operations result in sealing of the cells and vesicles. Loading in this way results in many normally sized cell membranes, while some smaller loaded vesicles are also formed. These vesicles may be purified by chromatography, centrifugation, or other means.

Increasing Vesicle Size by Heating

A surprising result occurred when loaded red cell were heated. The vesicles coalesced into larger vesicles, but did not lose their contents in the process. Presumably the membranes of adjacent vesicles first would touch, then fuse forming an intervesicle pore connecting the two. This pore then grew in size to allow the membrane to assume its lowest energy conformation which was a larger more spherical single vesicle. Interestingly, the sizes however did not increase indefinitely, but instead growth continued to about the size of the original cells (8 microns), then growth produced chains of connected vesicles, forming tubes and tubes with branches. This limitation in growth might be attributable to the cytoskeletal components of the red cells still attached to the inner surface of the membrane which could still exert a control on the curvature of the membrane. The fusion process did not apparently result in loss of the originally encapsulated or loaded material, since the fusion process did not breach the membrane so that the inside contents were not directly exposed to the solution outside the vesicles. This growth process increased with temperature and time, thus providing a method to control the end vesicle size and products formed. Rapid coalescence into 3 to 8 micron vesicles from smaller ones occurred at 100 degrees C. after 2 minutes, and tubes and branched tubes were also more produced at 100 degrees C. after 3-4 minutes. Lower temperatures, between 40 deg. C. and 99 degrees C. produced a slower rate of vesicle fusion.

Loaded Materials and Targeting Moieties

The cell vesicles may be loaded with almost any compound or particle (from 0.8 nm to 5 microns). These may then be used for imaging, therapy, controlled movement or sorting, or other applications. This specification discloses how to package such substances into complex biological membranes for a multitude of applications, and how to achieve loading of cells and vesicles with high concentrations of materials.

The vesicles can optionally be derivatized further by attaching either covalently or with binding ligands various materials to the outside surface of the vesicle for the purpose of targeting or changing the properties of the exterior surface. The material attached may be fluorescent, X-ray absorbing, magnetic (paramagnetic, diamagnetic, ferromagnetic, antiferromagnetic, superparamagnetic), nanoparticles, small molecules, proteins (antibodies, antibody fragments, single chain antibodies, enzymes, structural proteins), peptides, drugs, inorganic and organic molecules, organometallics, polymers, bacteria, and viruses.

Loading Cells other than Red Blood Cells

Other cells can be loaded by the techniques described. Certain cell populations can be isolated by cell sorting (e.g., fluorescent activated cell sorting, FACS), immunobinding of magnetic particles followed by magnetic isolation (then release of the magnetic beads), differential centrifugation, affinity chromatography, and other selection processes. Certain cell types can also be expanded ex vivo clonogenically using cell culture to produce additional cells. Using the described techniques, these specific cells or mixtures can be loaded. In some cases it is of advantage to create loaded vesicles that will now have the same surfaces as the starting cells. In vivo use can take advantage of the natural biodistribution of such cells (such as immune cells and platelets), while providing a way to modulate the pharmacokinetics of their distribution by varying their size.

Loading Bacteria and Viruses into Cell Membrane Vesicles

Bacteria and viruses, inactivated bacteria and viruses, or bacterial and viral components, are loaded into the vesicles by preparing a (usually) concentrated solution of the bacterial or viral material and subjecting it to the loading protocols described. This then “hides” the bacterial or viral material and permits their introduction into an animal. One use is so they will not be immunologically rejected, at least in the usual short time frame. Slow breakdown of the vesicles would initiate immune response, and this time release encapsulation breakdown would present more antigen over time so that booster shots could be avoided and the altered immunologic response would result in a more effective vaccine.

Novelty and Distinction from Prior Art

A number of other groups have loaded various materials into synthetic liposomes. These have a number of disadvantages for in vivo use: a) short blood half-lives, b) toxicity, c) immunogenicity, d) low loading of substances to be encapsulated, e) lack of water pores in membranes, which greatly lowers MRI signals for many contrast agents. Red blood cells have also been used to load substances. However, the loading was many times lower that the methods disclosed herein. Johnson et al. (Magn Reson Med. 1998; 40(1):133-42), Magnetic Resonance in Medicine 45:920-923, 2001) used hypo-osmotic shock, resulting in 28-30 mM Gd or Dy, but the novel methods disclosed here produced cells or vesicles loaded with 160 mM Gd (a factor of 5.3 times higher). Johnson et al. only describe hypo-osmotic shock for loading, which imposes severe limitations on the amount that can be loaded. For best sensitivity, it is well known that highest loading is desired, yet the method of Johnson limits the loading. It would have been desirable for Johnson to achieve higher loading, but no such method was described to do so because it was not obvious how to do so at the time. Furthermore, it is here described how to control the size of vesicles for various applications to control clearance and extravasation. Johnson et al. only loaded full sized red blood cells. In addition, the Johnson group did not teach nor demonstrate targeting although that would have been desirable. The loading of red cell vesicles with viruses and bacteria is novel and not previously taught. The implementation of multicolor MRI has not previously been described to our knowledge or achieved. The wide variety of useful applications presented herein is novel and not obvious to those skilled in the art because the novel agents to accomplish these applications were not thought of or available.

Pharmacokinetics

A distinct advantage of the disclosed method is the altered and controllable pharmacokinetics of the loaded material. A small molecule, such as a drug or contrast agent, will now have a tremendously different blood half-life. The removal of the cell-derived vesicles can be controlled by the size of the vesicles formed. Small vesicles will be removed more rapidly by the kidney. Since the drug or agent is encapsulated, the pharmacokinetics are no longer a property of the drug or agent, but are now determined by the cell or cell-derived vesicle. Different cells used will have their own biodistribution and fate. Different cells have different mobilities, surface properties, receptors, binding affinities, and localization patterns. The cell type most useful for a particular application can be chosen. Furthermore, the cells or cell-derived vesicles may be targeted as described below, thus also altering and controlling the pharmacokinetics of the encapsulated material. Cells and vesicles break down and are catabolized at various rates. The specific cell type and vesicle size can be chosen to program a specific lifetime for the encapsulated material, before it is released. Antigens or other agents may be incorporated into the cell or membrane surface that will also control the lifetime of the cell or vesicle. For example a foreign blood type antigen can be incorporated into the surface of the cells or vesicles during preparation or loading. Once re-injected, such cells will be targeted for lysis by the immune system, causing earlier breakdown of the cells or vesicles and release of the loaded material.

Magnetic Resonance Imaging (MRI)

Gadolinium is a preferred element for MRI due to its 7 unpaired electrons. Although gadolinium (Gd) by itself is toxic, it was found when highly chelated, e.g., to DTPA (diethylenetriaminepentaacetate) or DOTA (1,4,7,10-tetraazacyclododecane-1,4,7,10-tetraacetic acid) that it is then passivated and becomes tolerated in vivo. It is used in about 30% of MRI procedures. Some minor synthetic modifications have been made to the chelating moiety, but also with minor alterations in properties. Experimentally, Gd-DTPA has been coupled to albumin, antibodies, and other molecules, but useful products have generally not resulted. Although Gd-DTPA is a useful image enhancer, it has severe limitations. It is low in molecular weight (503) and leaks out of the vasculature very quickly. It also clears very rapidly through the kidneys. Its main strength is visualization of some brain tumors where the blood brain barrier prevents leakage, but where it does egress through the leaky tumor vasculature. It is not a blood pool agent useful for imaging blood vessels due to its short blood residency.

The present disclosure provides a method of increasing blood residence time without increasing toxicity. Cells or vesicles are loaded with contrast agent and then injected into the subject to be imaged. In a preferred embodiment, blood is taken from a patient, the red cells pelleted, mixed with concentrated Gd-DTPA or other contrast media, forced through a filter, and then reinjected into the patient. This is a simple and straightforward procedure that can be automated, making it clinically feasible. Although other methods have been previously described for loading contrast material into vesicles or cells, the method disclosed here is far simpler and was found to be surprisingly effective. Blood cell vesicles prepared this way showed extremely high vascular imaging 10 to 15 minutes after injection. Tumors were also highly contrasted. Clearance was mainly through the kidney, but delayed longer than free Gd-DTPA. Excellent contrast developed in the heart, heart vessels, lungs, liver and other organs, much more so than with Gd-DTPA alone. For comparison, the same standard suggested doses of Gd-DTPA were used for both the free Gd-DTPA imaging and the Gd-DTPA prepared using blood. A number of advantages of this method are apparent: 1). There is no toxicity. The patient's own blood may be used, and FDA gadolinium chelate is used at the recommended non-toxic dose. Even if the vesicles break down, the products are all non-toxic. 2) A true blood pool agent is immediately created. This is a novel and non-obvious achievement since it has been difficult to obtain by other methods, as evidenced by the absence of an FDA approved agent, even after about 20 years of research. 3) By attaching antibodies, peptides or other targeting moieties, a large payload of imaging agent can be delivered to the specific site of interest. 4) By varying the size of the red cell vesicles, blood half life and extravasation rates can be controlled. 5) “Multicolor MRI” can be achieved by separately loading e.g. Gd, Dy, Fe, Co particles, or other contrast agents into vesicles then linking different antibodies to each. The cocktail is injected and each type of vesicle can be recognized from its distinct MRI signature. 6) The whole process of loading and even antibody labeling can conceivably be done in less than 30 min., so that a patient could be imaged after a simple blood draw. 7) The vesicles are not immunogenic.

Although Gd-DTPA was used in the above description, any other contrast agent may be used, including other gadolinium compounds, iron particles, manganese agents, dysprosium compounds, or cobalt or nickel-containing materials.

Proton Exchange Rate—Volume Fraction

It is here disclosed a surprising observation that MRI contrast agents encapsulated in red blood cell membranes give a much higher signal than the same agents encapsulated in a synthetic liposome of the same size. This large difference might be explained by a subtle but important point for MRI: the exchange of water protons near the contrast agent with the environmental water. Red cell membranes have many aquaporin water channels and the mean residence time of water inside the blood cell is about 10 msec. with a water permeability (Pd) of 6×10⁻³ cm/sec at 37° C. This means that the contrast agent in the red cell membranes has access to the surrounding tissue volume. If liposomes were used that were impermeable to water, the volume fraction of water of the vesicles in tissue would be proportionally (greatly) reduced, thus diluting the contrast signal. The use of red cell membranes is therefore not just a convenience, but important for increasing the signal of T₁ reagents.

For example, if a vesicle contains 150 mM Gd (which we estimate to have obtained), T₁ in water is:
1/T₁=1/T₁initial+r1C
for Gd-DTPA, r₁=4.3 (mM-sec)⁻¹. For T₁initial (water)=2.5 sec, this yields a T₁=645(sec)⁻¹. If the volume fraction of the vesicle is 1%, and water freely exchanges, then the effective r₁ is:
r₁=0.99×1/(2.5 sec)+0.01×645(sec)⁻¹=6.87(sec)⁻¹
The T₁ signal is proportional to (1-e^{-TR/T1}). The difference before and after contrast agent will be:
ΔI=(1-e^{-TR/T1}″) with agent—(1-e^{-TR/T1}) for water
For a usual value of TR=500 ms, this gives:
ΔI=0.968−0.181=0.787
On the other hand, if the water does not exchange (as in a liposome without water channels), the signal seen will be from the bulk water (99%) plus the signal from the liposome. Subtracting the before (100% water) image gives a contrast proportional to:
ΔI=0.01×1(for liposome)+0.99×0.181−0.181=0.0082
The image contrast difference between these two cases is therefore:
0.787/0.0082=96
The water porous vesicles of this disclosure would then give a signal 96 times greater than use of an impermeable liposome. This observation distinguishes this novel approach from liposome encapsulated contrast agents.
Molecular Multicolor MRI

In vivo molecular imaging, i.e., using targeted contrast agents to molecular markers, is now within the realm of feasibility. Work by many researchers has begun to identify unique or highly expressed molecules on aberrant tissue, such as various cancers subtypes. These molecular markers can be targeted with drugs for a more specific therapy with fewer side effects. For example, overexpression of the tyrosine kinase Her-2/neu on certain (˜30%) breast cancers can now be treated with a monoclonal antibody (Herceptin) that binds to and inactivates this growth factor receptor. Similarly, epidermal growth factor receptor (EGFr) is overexpressed in many tumors types including gliomas, prostate, colorectal, squamous cell and other carcinomas, and a therapeutic antibody (Erbitux [Cetuximab]) is now available for treatment. Candidates for these therapies are currently evaluated by biopsy. A less invasive and more complete method (visualizing the whole tumor) would be in vivo imaging, since the morphological distribution and response with therapy could be more easily ascertained. It would be useful for identifying and monitoring patients with sufficient receptor overexpression for personal-tailored therapeutic interventions, and also for depicting tumor tissue and determining the currently largely unknown heterogeneity in receptor expression among different tumor lesions within and between patients. Because multiple conditions must be distinguished, it would be desirable to have separate signals to report on different molecular targets, or molecular “multicolor” MRI. Here “multicolor” refers to multiple distinguishable signals, and not actual colors in the visible spectrum. During an MRI exam, it would be desirable to have several potential targets identified with different specific agents. For example, multiple tumor types could be probed to correctly diagnose an individual's condition an prescribe the best therapy. Until now only single functional contrast agents have been described. Here we disclose methods to introduce multiple distinguishable contrast agents for imaging.

In one embodiment, three compounds can be used for molecular targeting: Gd-based (gadodiamide), Dy-based (dysprosium-diethylenetriaminopentaacetic acid-bis-methylamide-Dy-DTPA-BMA), and Fe-based (monocrystalline iron oxide nanoparticles-MION-Fe₂O₃). Three separate vesicle preparations are loaded with one of these agents, and the vesicles are derivatized with three separate antibodies, so that each type of vesicle will target a specific tumor type. The mechanism of action for all of the above compounds is based on their ability to catalyze NMR relaxation properties of water protons in a concentration dependent manner. However, the Gd-based compound is primarily a T₁ agent, so short T_{R} sequences will be used, as is common for this agent. The Dy-based compound has weaker dipolar effects and stronger susceptibility effects than does the Gd-based compound, and is detected primarily through its ability to relax water protons by T₂* susceptibility effects. Iron oxide particles are superparamagnetic and have high magnetic susceptibility (100 to 1000 times stronger than paramagnetic substances) and create a relatively large regional gradient magnetic field. Such a gradient readily influences water molecules diffusing close to the particles, reducing T₁ and T₂. When water protons diffuse through this inhomogeneous magnetic field, variations in the Larmor frequency result and phase synchrony is lost decreasing transverse magnetization and shortening T₂. Unlike T₂* signal losses, the resulting T₂ signal losses can not be recovered with spin echo refocusing strategies. Pulse sequences sensitive to T₁, T₂ and T₂* are used to distinguish the Dy, Gd, and Fe compounds. Because T₁, T₂, and T₂* effects are present for any agent, there will be some overlap in trying to absolutely distinguish multiple reagents and concentrations. However, analogously, two compounds that have spectral overlap at two different wavelengths can be completely distinguished by two separate measurements and solution of the simultaneous equations. This strategy can applied to multiple signals, and is commonly used in fluorescent imaging to distinguish 24 fluorophores in spectral karyotyping. Similarly, it is possible to achieve good distinction between different agents by a similar analysis of data taken with different pulse sequences. For example, T₁ agents (such as Gd) usually have transverse to longitudinal relaxivity (r₂/r₁) ratios of ˜1, whereas this ratio for iron oxide particles is 10 or more. By constructing the T₁/T₂ ratio, these two agents can be distinguished. Other distinguishable contrast agents may also be used for more “colors” including compounds containing cobalt and nickel.

X-Ray Contrast Agents

Similar to contrast agent development for MRI, there have been few new agents approved by the FDA in the past 25 years. Iodine is inexpensive and heavy enough to absorb X-rays, so is almost exclusively used. Barium is used for the alimentary tract, but is too toxic for intravenous use. The few approved iodine agents are basically tri-iodobenzene derivatives, with groups added for water solubility. One improvement was the formation of dimers of these compounds to reduce osmolality and concomitant patient pain. Similar to the MRI agents, such as Gd-DTPA, the molecular weight of the iodine compounds used are very low. For example, one of the most commonly used agents is iohexol (N,N′-bis(2,3-dihydroxypropyl)-5-[N-(2,3-dihydroxypropyl)-acetamido]-2,4,6-triiodo-isophthalamide, Omnipaque) which has a molecular weight of 821 Daltons. This quickly exits the vasculature, and can only be imaged for a very short time. This generally necessitates catheterization, where a catheter is snaked through an artery to the hilus of an artery and the dye is injected and immediately visualized by X-ray fluoroscopy. Such a procedure is both risky and expensive. A longer blood residence agent would be of great value, but so far no such agent is FDA approved, despite many years of diligent research. Many other trial agents that have different pharmacokinetics have proven either too toxic or do not clear the body satisfactorily. Here we show how the method disclosed easily overcomes these difficulties and achieves the much sought after goal of an effective and non-toxic blood pool X-ray contrast agent.

In a preferred embodiment, a small amount of blood is taken from the patient, centrifuged, the cell pellet mixed with an iodine contrast agent with attention to osmolality (such that the resulting vesicles do not burst when reinjected), the mixture is forced through an appropriate sized filter, and the sample injected into the patient. Variations of the procedure can be done as described earlier, such as additional washing of the blood, use of other methods for loading, further filtering to refine the vesicle size, and separation of the loaded cells or vesicles from free agent. The latter separation may not be required for an application, since the free iodine agent quickly dissipates from view. Once again, the final product is non-toxic, since the amount of iodine agent used is within the recommended and approved safe dose, and the patient's own blood is not toxic.

The method may also be used to load other types of X-ray contrast material into cells or vesicles, such as gold, tungsten, bismuth, or gadolinium nanoparticles or compounds.

The concentration of contrast agent or therapeutic agent incorporated into the cells or vesicles is usually important, i.e., the more the better, since this gives more signal or dose per vesicle. Gold nanoparticles and other agents can be isolated in pure water or solvents or in low molarity salts, then concentrated by drying or partial drying. When the vesicles are formed or mixed with these dried or partially dried agents, the incorporation concentration can be substantially increased.

Coronary, Carotid, Renal, and other Artery Imaging

Coronary vessel imaging is of great concern due to the number of heart attacks per year. One would like to know the condition of the coronary arteries: are they stenosed, is there atherosclerotic plaque, and is the plaque vulnerable to rupture, which would initiate a myocardial infarction? Currently there no adequate methods for screening patients. Use of blood tests measuring cholesterol and low density lipoprotein are very indirect and do not adequately indicate a possible impending and critical problem. Ultrasound has poor resolution, and while useful for checking heart valve function, it cannot assay the anatomic or physiologic condition of the coronary arteries. Stress tests also have low resolution and cannot directly delineate the condition of the coronary arteries. Catheterization does show the anatomic condition, but is both risky and expensive, and is only done if critical signs are evident. A non-invasive method to assay the coronary arteries is sorely needed. The method disclosed here is capable of fulfilling this need. In a preferred embodiment, a small amount of blood is taken from the patient, it is centrifuged and the cells are mixed with an X-ray or MRI contrast agent. The cells are forced through a filter, or loaded vesicles formed by the other means described herein, and the product reinjected. The loaded cells or vesicles provide high contrast imaging of the coronary arteries for an extended period of time that can be easily visualized by X-ray CT or MRI. These 3-dimensional imaging methods reconstruct accurate voxel concentrations and are not confounded by agent in other areas, such as would occur in a simple 2-D projection image. With rapidly acquired single images or combination of images taken with EKG gating and image alignment, to overcome the motion of the beating heart, the extent of stenoses and plaque development can be determined.

The distinguishing of vulnerable plaque from stable plaque is a further objective in arterial imaging in order to assess the risk factor if plaque or a stenosis is anatomically detected. Vulnerable plaque is characterized by a high fat content, high vascularity, angiogenic activity, fibrin deposits, and high oxidized LDL content. Calcium deposits were once thought to indicate risky plaque, but further studies showed this was poorly correlated. The methods described will permit assessment of vulnerability since the vascularity of the plaque can be measured with a blood pool agent. An index of vascularity per volume of the detected plaque can be generated. An additional approach is to us a small vesicle size that selectively leaks out of leaky angiogenic endothelium. This agent would assay the angiogenic activity, and hence vulnerability, of plaques. Further distinction of plaque composition and vulnerability can be achieved by targeted liposomes to molecular plaque markers, such as oxidized LDL or fibrin.

Although a simple preferred embodiment was stated, the other variations described may be used, or those variations obvious to one skilled in the art, which in some cases could provide improved imaging and detection.

The above discussion focused on coronary imaging, but most of this applies to assessment of the carotid and other arteries for stenoses and vulnerable atherosclerotic plaque. Also, any imaging done with invasive catheterization, such as coronary, cerebral, and renal, could now be achieved by non-invasive imaging of the intravenously administered agents of the present invention.

Other Imaging and Detection

Since the disclosed methods may be used to load many types of compounds, biomolecules, drugs and agents into cells and cell-derived vesicles, materials can be loaded that would enhance other forms of imaging, including, but not limited to: X-ray, MRI, PET, SPECT, visible light, infrared, fluorescence, Raman scattering, light and electron microscopy, spectroscopies, backscattering, and ultrasound. Materials useful for these other forms of detection include molecules or particles useful for X-ray absorption containing elements including but not limited to: gold, platinum, iodine, lead, iridium, osmium, tungsten, bismuth, cesium, barium, uranium, gadolinium, europium, the lanthanides, the actinimides, and silver; molecules or particles useful for MRI containing the elements including but not limited to: gadolinium, dysprosium, iron, cobalt, and nickel, or molecules with distinctive relaxivities, such as fats; molecules or particles useful for PET containing positron emitting elements including but not limited to: carbon-11 and fluorine-18; molecules or particles useful for SPECT containing radioactive elements including but not limited to: iodine, indium, or technetium; molecules or particles useful for visible light detection that are colored or absorptive in the visible wavelengths; molecules or particles useful for fluorescent detection including but not limited to: fluorophores, quantum dots, and phosphors; molecules or particles useful for Raman scattering including but not limited to: organic molecules and metal particles; molecules or particles useful for electron microscopy containing the elements including but not limited to: gold, silver, uranium, tungsten, bismuth or vanadium; molecules or particles useful for ultrasound detection including but not limited to: gas bubbles of various gasses.

Targeting

The methods described of loading cells and vesicles with materials and administering them (either intravenously, intra arterially, intramuscularly, intraperitoneally, orally, or other means) will have biodistributions determined by the cell type, the size of the cell or vesicle, the homogeneity of the sample or mixture, the mode of administration, and the time after administration. As discussed, this already provides methods to control the localization to various tissues and organs, and thus provides a level of targeting. An additional powerful mode of targeting is based on inclusion of an antibody, antibody fragment, single chain antibody, peptide, drug, compound, ligand, substrate, or other material that binds to specific sites. Such directing moieties can enhance delivery and specificity.

In the present invention, the loaded cells or vesicles may also be further directed to specific sites by attachment of binding molecules to the cell or vesicle surface. Conventional methods may be used, such as chemical crosslinking of the binding molecule to the membrane surface. However, more specific methods are here disclosed that are particularly relevant to the loaded cells or vesicles, that provide rapid and straightforward means to attach the binding molecule. Such rapid and efficient methods are important in making the procedures useful clinically.

The first method is to prepare a conjugate of an antibody to a component of the loaded cell or cell membrane that is coupled to the binding molecule to the desired target. An example is a bifunctional antibody to band 3 (an anion channel) or glycophorin with its other half being an antibody to the desired target, such as EGFr (epidermal growth factor receptor). After or during loading of the cells or vesicles, they are exposed to this chimera and the cells or vesicles quickly become covered with the bifunctional antibody. Excess can easily be removed, if desired, by centrifugation, dialysis, filtration, or other methods. When the loaded and labeled cells or vesicles are injected, they will now bind specifically to cells expressing EGFr.

A second method to attach a targeting moiety is to first link the binding molecule to a polymer or other agent that effectively adsorbs or binds to the cell or vesicle membrane. For example, a dextran polymer was derivatized with amino groups which were then covalently linked to an antibody. This dextran-antibody complex was found to tightly bind to erythrocyte membranes, making them target the antigenic site. Many other polymers may be used such as polylysine, amino derivatized dextran, ficoll, and proteins.

A third method is to first derivatize the antibody or binding molecule with a lipid moiety, such as palmitoyl chloride. This introduces a hydrophobic region into the targeting substance. The conjugate is introduced before the cells are loaded, and during the loading procedure, the cell membranes are disrupted, allowing efficient fusion with and incorporation of the lipophilic substance during the phase where the membranes are torn or disrupted.

A fourth method is similar to the previous method, but uses a targeting molecule that is already hydrophobic or has a hydrophobic region (such as a membrane protein or hydrophobic drug). During loading, the cell membranes are disrupted, allowing efficient incorporation of the lipophilic substance during the phase where the hydrophobic part of the cell or vesicle membrane is more exposed and accessible to lipophilic material. Lipophilic moieties may also be inserted into intact or relatively intact membranes, and hence a lipophilic targeting moiety may be applied to the cell or vesicle at any stage of handling, thus incorporating the targeting molecule.

A fifth method is to covalently link the targeting molecule onto the membrane surface. For example, antibodies may be activated with a bifunctional crosslinker, or other chemical modifications to introduce reactive groups on either the membrane, the targeting molecule, or both, such that when they are incubated together, covalent bonds are formed between them.

A sixth method is to use non-covalent adsorption. Many binding couples such as avidin and biotin, zinc fingers, and other stable molecules or moieties may be used. Even electrostatic (charge), van der Waal's, hydrophobic and other force interactions between the membrane or membrane components and the targeting molecule or a derivative thereof, may be utilized to couple the targeting molecule.

In addition, other targeting moieties and coupling methods known in the art may be used.

Targeting Using Magnetic Localization

The disclosed method that permits loading of cells and cell-derived vesicles with compounds, proteins, contrast agents, particles, and other substances, can be used to encapsulate magnetic particles and nanoparticles in the size range 1 to 5,000 nm. These may then be used to isolate, identify, assay and select loading, or purify the product. The cells or vesicles can then be targeted to a specific region by use of magnetic fields produced by permanent or electromagnets.

An example of the benefits of this approach is shown for adoptive immunotherapy. In this approach, killer T cells that can destroy patient tumor cells are removed from the patient, isolated, and grown ex-vivo to high numbers and injected back into the patient. A current obstacle is the poor localization of the killer T cells to the tumor, resulting in low benefit to the patient in many cases. In the method disclosed here, the ex-vivo proliferated cells may be loaded with magnetic nanoparticles before injection into the patient. Subsequently, they may be concentrated in the tumor region in much higher numbers by small magnets placed in the tumor or external permanent or electromagnets. The greatly enhanced number of lymphocytes at the tumor will result in better efficacy.

Magnetic localization can be used for other cell types and purposes, such as bringing appropriate cells in higher numbers to an infection to more effectively fight it. In this way, for example, gangrene can be more effectively treated to avoid amputation. The vesicles may be loaded with antibiotics or other drugs that would then be more effective when targeted due to their higher local concentration and avoidance of systemic toxicity by reducing the concentration in unwanted tissues.

Drug Encapsulation and Gene Therapy

By the methods disclosed, drugs may easily be encapsulated into normal body cells or cell-derived vesicles. These may also be targeted to specific sites by the methods disclosed. The pharmacokinetics of the drugs will be completely different using the methods disclosed, and enable the property of the drug to be separated from its native pharmacokinetics. Small molecule drugs frequently have the problem that they clear the system too rapidly, diffuse out of the vasculature, and have pharmacokinetics that prohibits their effective use. A major problem is the low concentration of the drug at the desired site and systemic toxicity. Modification of the drug itself to control its biodistribution and clearance often leads to inactivation or loss of the drug properties. By the methods disclosed, drugs may be encapsulated in their most effective form, with no further design changes, and delivered by controlling the cell type used, vesicle size, and targeting component.

Gene therapy requires that nucleic acids be delivered to and transfect deficient target cells. The vesicle methods described may be used to encapusulate nucleic acids for transfection and improve their efficacy by targeting the vesicles to the cells of interest. Additionally, the vesicles may be loaded with substances known to enhance transfection efficiencies, such as positively charged lipids, calcium phosphate, cations, translocation sequences, cationic gold particles, and other such enhancers.

It is an object of this invention to treat by the above disclosed vesicle mediated gene therapy and other aspects of this invention, diseases or conditions known to be caused by genetic mutation where a gene is either missing or non-functional, in which case it can be restored, or aberrant and overactive, and can be downregulated, both by transfection of a new gene or genes that replace the missing function or produce inhibitors of the overactive gene. Such conditions include diabetes, where islet cells do not respond to glucose to produce insulin, Parkinson's disease, where there is a lack of dopamine production, cystic fibrosis, where a single gene is defective causing lung failure, cancer, where genetic mutations have removed control of cell division, and many other conditions.

Controlled Release of Drugs by Vesicle Disruption

As described above, almost any drug or agent can be encapsulated by the disclosed methods inside red cell membranes, or those of other cell types, or synthetic vesicles. However, it is an object to deliver such drugs to a specific target within the body, or in other applications to a specific site, and then release the agent. The vesicles may be targeted by one of the means described, so that the vesicles are delivered to and bind to the desired target site. Now, however, the vesicles need to open to release their drug contents. Alternatively, the vesicles do not have to be pre-targeted, but may be forced to release their contents as they pass through the treatment region. The vesicles or cells may simply circulate or passively diffuse or pass through the desired treatment volume. Energy applied to the region can cause the vesicles or cells to release their contents. In this way, the localization to the treatment volume is achieved by the energy delivery rather than specific targeting of the vesicles or cells.

Because the cells or vesicles, which are biocompatible, normally break down slowly, they may be used as time release vehicles for a drug.

Another method, here disclosed, is to use vesicles from a different individual or species. These vesicles will have a limited lifetime in the recipient due to immunological rejection. In detail, one mechanism of the immune response is that killer T cells will actively break down the vesicles. Another mechanism is the complement system which creates holes in the foreign cell membrane. These and other rejection responses cause the vesicles to break down and release their contents into their environs. Because these responses are not instantaneous, and can be controlled by immunization and other modulating tactics, such as antibody neutralization or immunosuppressents to delay the response, the vesicles can have time to first target their intended site before the local release of their drug or other cargo.

A blood cell may be used that has a different blood type, or a foreign blood cell. Alternatively, vesicles with inserted immunogenic or foreign material may be used. In these cases, the complement system would be activated, and after a certain response time, the cells or vesicles would be breached (the complement system creates holes in the membrane), and the contents released.

Foreign blood cells need not be used. A patient's own blood may be treated with an agent that elicits an immune or inflammatory response. For example, a sample of patient's blood, removed for processing, could be treated with anti-human red blood cell membrane antibodies raised in rabbits, or mouse, or other species. These would then make the cells membranes targets for the immune or complement system when reinjected into the patient. Similarly, chemicals or other biochemicals may be bound to the cells or vesicle membranes that in turn will stimulate a biological response resulting in membrane disruption.

In another embodiment, vesicles loaded with magnetic particles are first targeted to the desired site by an antibody, peptide, magnetic attraction, or other targeting method. Alternatively, the vesicles are acted upon while just passing through the target volume. An alternating electromagnetic field is then applied, causing the magnetic particles to mechanically rupture the membranes, thus releasing the internalized drugs or cargo. Similarly, loading with other materials such as microwave absorptive particles can be used to locally heat the vesicle causing release of its contents. Ultrasonic, radiofrequency, microwave, infrared, or other externally applied energy may be used to heat the vesicles or their contents including gases or liquids that become gases that will then expand or react in such a way to disrupt the vesicles. The external energy may also be used to mechanically disrupt the vesicles.

Another method for timed release from the vesicle is to encapsulate an enzyme that will break down the vesicle membrane. For example, a lipase or protease loaded into the vesicle would act to disrupt the membrane, thus effecting the release of its contents. Such an enzyme can be controlled by a number of means so that it would cause drug delivery at an optimal time. For example, vesicles loaded with a drug could be targeted to a tumor, then opened to release a chemotherapeutic agent. The lipase activity can be controlled by loading it into the vesicles just before administration to the patient, or using multilamellar vesicles that take the enzyme longer to digest. Encapsulating additional enzyme substrate (for example protein or lipids) would slow the attack on the vesicle membrane and would allow programmable time delays for when the average time of vesicle disruption would occur. When the membrane is breached, the enzyme would be released into the blood, but would cause little further normal tissue damage since it will be quickly diluted and there are many enzyme inhibitors and proteases already in the blood that would inactivate it, for example by alpha-2-macroglobulin. A drug or compound may also be used to breach the membrane after a delay. For example an acid , base, detergent, caustic agent or other substance capable of eroding the membrane may be loaded into the vesicles such that they will subsequently be disrupted causing release of the contents. Binders, polymers, smaller vesicles, or other compounds that temporarily inhibit or restrain the membrane-disruptive agent may be used to effect delayed release of contents from the primary vesicle.

A two step mechanism for vesicle release is also disclosed where first the vesicles are administered and targeted, and in a second step, another agent that interacts with the vesicles is then administered that causes the vesicles to release their contents. For example, a novel antigen can be incorporated into the red cell vesicles being prepared ex vivo. After antibody or other targeting to the desired site is optimally reached, another ligand that binds to the novel antigen is administered that the patient is already primed to reject. For example rabbit antibody to the novel antigen would target the vesicles, but then would be recognized by the immune and complement system and macrophages that would then attack and lyse the vesicles, releasing their contents. The antigen need not be novel. For example, a normal protein can be attached to the vesicle surface, such as collagen, nuclear lamin, DNA, intracellular proteins, or many other common body components that are not in or exposed to the blood. Being normal body components, they would not elicit any immunological activity or response. However, because they are not normally in the blood, one may then in a second steip introduce into the blood an antibody to this material, which would then bind the pre-targeted vesicles. If this antibody was raised in another animal, it would elicit an immune response resulting in disruption of the vesicles, causing their contents to be released. However, this antibody could be humanized so that by itself it would not cause any immune complexes in the blood, but when it bound to the target vesicles it would elicit a response from the complement system resulting in disruption of the vesicles.

In another embodiment, an antigen is attached to the vesicle or cell membranes. Attack by the complement system is delayed by one of two methods: a) the antigen is buried or covered with another substance, and this substance or substances can be layered. The covering substance would then be removed either by desorption or slow dissolution, or it could be a substrate for enzymes in the blood or administered subsequently. The antigen would then after a programmable period be exposed to elicit a response from the immune or complement system resulting in disruption of the membrane and release of the contents. b) Alternatively, the antigen is covered by an antibody fragment, such as Fab or ScFv. The binding affinity of this fragment can be selected to be weak through strong, thus programming how long the antigen is covered. Antibody fragments contain no Fc region, and therefore do not activate the complement system. Once the antibody fragment dissociates, which it will at some point since it is not covalently bound and has a certain off rate, the antigen would be exposed and will stimulate an immune and complement reaction, resulting in breakdown of the membrane and release of the vesicle contents. A further method is to subsequently administer a whole antibody (containing the Fc region) to the antigen that would either displace the antibody fragments, or bind to the antigen after the fragments had dissociated. This administration of whole antibody would then serve to elicit the complement lysis of the cells or vesicles, and the time of lysis would be controlled by the administration of the whole antibody, which could be done after the vesicles or cells were localized to the target region.

For the two step process, where an antibody to initiate complement lysis is administered after targeting of the vesicles, IgGs and other immunoglobulins can be used to stimulate immune responses and the complement system. However, IgM is the most potent isotype stimulator of the complement system, and its use would generally be preferable, or this fact considered in therapy design. IgM is not typically used in therapies, such as antibody therapies to treat cancers, since IgM extravasates less efficiently from the vascular system due to its larger size than IgG. In such therapies, the antibody must escape the vascular compartment to reach and bind to the tumor cells. However, in the case of lysing cells or vesicles already in the vascular compartment, as disclosed here, this restriction is lifted, and the improved effectiveness of IgM in stimulating cell breakdown after binding may be utilized. Covalent linking of C3b to IgG enhances stimulation of the complement system over IgG alone, and these complexes may be used to advantage.

Use of the complement system has additional advantages, such as: release of factors C3a and C5a that cause increased permeability of blood vessels for better permeation of drugs or agents to reach target cells (such as tumor cells), C5a also acts as a chemotaxis agent to attract macrophages, C3b targets cells for phagocytosis, the immune system is stimulated, for example by breakdown of C3b to C3d that binds to antigens and enhances uptake by dendritic and B cells.

The complement system involves many components and modulators, and for the purpose of controlled lysis of the cells or vesicles carrying the cargo, it is disclosed that these components can be regulated. For example, autologous cells contain Decay Accelerating Factor (DAF) and CR1 on their surface that inhibits both classical and alternative C3 convertases. Other factors also inhibit complement activity, such as Factor H, Factor I, C1 inhibitor, C4bp, MCP (membrane cofactor protein), S protein, SP-40, HRF (homologous restriction factor), MIRL (membrane inhibitor of reactive lysis, or CD59), and sialic acid. While the red cells are being loaded ex-vivo, these proteins may be inactivated by binding specific antibodies to them or use of drugs or protease inhibitors. Inhibitors may also be depleted by heating the cells or vesicles to denature them (to 40 to 100 decrees C.), or treated with enzymes to inactivate or remove them (such as sialidase (neuraminidase), trypsin, pepsin, and proteanase K). This inactivation may also be partial to produce a longer lived membrane before the Membrane Attack Complex (MAC) forms, which results in release of the cell or vesicle contents. Other modulators of the complement system in the serum may also be temporarily cleared or altered by administration of drugs, antibodies, or other specific inhibitors.

The classical and alternative complement pathways may also be controlled to enhance vesicle lysis. In one embodiment, it is advantageous to throttle down the alternative complement pathway, so that lysis does not proceed without an antibody stimulus. The lysis of the vesicles will then be controlled by the administration of antibodies that bind to the vesicles. The alternative pathway can be down regulated by interfering with its necessary components, for example, by administering an antibody to factor B to inactivate it. In a more extreme case, the complement system can be more widely inhibited so that the lysis of the vesicles would then be controlled by administration of the required complement components.

Related to the complement system is the antibody-dependent cellular cytotoxicity (ADCC) mechanism. This is initiated by binding of antibodies to antigens on the cell or vesicle which then stimulates its breakdown mediated by destructive cells with Fc receptors, such as macrophages, neutrphils, mononuclear phagocytes, and natural killer (NK) cells. In addition to this innate immune system, the adaptive immune system may also be utilized for cell or vesicle lysis. In this case, the host is primed with an antigen and produces antibodies and cytotoxic T lymphocytes (CTLs) against that antigen. When a vesicle is then introduced with that antigen, the CTLs have the capacity to break down the vesicle and release its contents. An antigen can be introduced into the vesicle during its ex vivo preparation.

In another embodiment, the vesicles are treated to render them less or more stable. In such a strategy, their lifetime in vivo will be reduced or extended, and therefore the average time before breakdown and release of contents can be controlled. As an example, it is disclosed that treatment of red cell membranes in low ionic strength causes increased fragility, perhaps due to elution of additional structural proteins. Chemical agents may also be used, such as crosslinkers and membrane insertants. For example, the crosslinker glutaraldehyde stabilizes the membrane and increases the time for its breakdown. Amphiphiles, lipids, fatty acids, surfactants, detergents, and lipophilic compounds can insert into the membrane and alter its properties, including stability. Various drugs, biomolecules, and other agent may also be exploited to alter the stability of the membrane.

Magnetic Localization

Magnetic nanoparticles have been localized by a magnetic field. This approach has two significant drawbacks: 1) the tiny magnetic nanoparticles are not strongly attracted to the field, and 2) the field draws the particles to the skin or to where the magnetic pole is, thus hindering or prohibiting effective localization to a deeper region, e.g., to an internal tumor. The present invention overcomes both of these drawbacks.

Magnetic nanoparticles for drug delivery or for delivery of magnetic particles themselves (which could then be heated for therapeutic effects), are problematic due to their small size. Magnetic particles above the magnetic domain size (typically 50-100 nm) can then be ferromagnetic and have a residual magnetization after a field is applied. Large ferromagnetic materials, such as iron filings, have the advantage that they are strongly attracted to a magnetic pole. However, ferromagnetic materials and particles can have severe disadvantages for human use. Because they have residual magnetism, they attract each other and will aggregate. These aggregates, particularly in the blood, would cause emboli, such as in the lung and brain and be very toxic. A second potential disadvantage is the large size of ferromagnetic particles, which could cause circulatory problems or would generally be rapidly cleared by the reticuloendothelial system that removes blood particulates. The use of small magnetic particles below the domain size results in their classification as superparamagnetic, namely that they do not retain a magnetization after a magnetic field is removed. These have the advantage that they do respond to a magnetic field, but do not become little magnets after the field is removed, and therefore do not then aggregate, at least for magnetic reasons. Unfortunately, even in a strong magnetic field, a suspension of superparamagnetic nanoparticles (a “ferrofluid”) is only weakly attracted and moves poorly towards a magnetic pole. This is because the particles are in suspension by Brownian motion and the thermal energy of collisions causes their easy reorientation and diffusion, negating their alignment for effective attraction.

Surpringly, when a ferrofluid was loaded into vesicles, it was found the vesicles became strongly attracted to a magnetic pole, whereas the ferrofluid itself was poorly attracted. For the ferrofluid by itself, which was colored, no localization at a magnetic pole could be observed by eye, even after minutes, but when a fraction of the same ferrofluid was loaded into the vesicles, the same field cleared all of the vesicles from the solution in a few seconds. When the field was removed, the vesicles were not magnetized and did not aggregate, validating that they retained the superparamagnetic property. This significant new behavior has important implications for magnetic localization, since the properties are not a simple addition of the ferrofluid plus the vesicles. For vesicles carrying drugs, or simply for the delivery of magnetic material, the greatly enhanced magnetic properties of ferrofluids loaded into vesicles is a significant improvement.

A second current problem with magnetic localization is that ferromagnetic or superparamagnetic particles are drawn to a magnetic pole, and cannot be arbitrarily focused to an arbitrary 3-dimensional position. For example, in human use, magnetic particles in the blood can be drawn to a magnet placed outside the body, but the particles will concentrate closest to the magnet pole, namely near the skin. It has not been found how to focus the particles to a deep internal region, thus limiting magnetic delivery, since most medical problems that need improved treatments are internal. Here we disclose methods to overcome this restriction.

In one embodiment, magnetic particles or particles in vesicles are administered into the body. An alternating magnetic field is applied using pole pieces placed on opposite sides of the region to be targeted, for example, on opposite sides of the abdomen or head, or on opposite sides of an arm or leg. This alternating field traps circulating magnetic particles in a region roughly defined by lines drawn between the two poles. By varying the shape of the pole pieces, the shape of the region can be controlled; for example the pole pieces can be pointed, defining a roughly cylindrical volume through the tissue, or the pole pieces can be opposing rectangular shapes, thus roughly confining the particles to the shape determined by imaginary lines connecting the two opposing rectangular pole pieces. Thin rectangular pole pieces would produce a slice. In this design, the particles are therefore not simply drawn to the skin, but are distributed throughout the field between the pole pieces which is controlled by the shape of the pole pieces. This design does not achieve focused 3-dimensional localization, but does enable localization throughout the tissue between the pole pieces, including deep regions, and permits shaping of this volume. By judicious placement of the rectangular, cylindrical, planar, or other magnetic localization volume, sensitive structures or tissues that should be avoided can be placed outside the volume. Treatment by then heating the magnetic particles, releasing drugs, inducing emboli, enhancing radiation, or other modalities based on the localized particles or vesicles could be achieved at depth while sparing normal tissue.

A 3-dimensional treatment volume can be achieved with the above strategy by first localizing or trapping the magnetic particles or vesicles in the volume between opposing pole pieces of an alternating magnetic field. The treatment modality is then applied from another direction (e.g., perpendicular), thus forming an intersection volume of treatment. For example, vesicles loaded with superparamagnetic particles and gold are localized to a deep tumor (e.g., pancreatic) by placing circular pole pieces of an electromagnet on opposing sides of the abdomen such that the tumor lies on an imaginary line connecting the two pole pieces. When a sufficient alternating magnetic field is applied, magnetic vesicles will be trapped along an approximate imaginary tube connecting the two pole pieces. Although the vesicles will be approximately throughout this volume, including some near the skin, x-ray radiotherapy can be directed perpendicular (or some other angle) to the pole pieces and confined to the tumor area as seen from the x-ray direction. The radiotherapy will be enhanced by the presence of gold. In this way, a 3-dimensional treatment volume can be achieved. The off-magnetic axis application of energy can be ultrasonic, infrared, microwave, radio frequency, light, or other source. In fact, a second magnetic off-axis field can be applied to heat particles or vesicles or disrupt vesicles so their contents are released.

The pole pieces may also be moved or scanned to create a larger region of confinement.

In a second embodiment, the above described alternating field using opposing pole pieces can be rotated relative to the target, resulting in concentration of magnetic material near the center of rotation. For example, let us assume two small circular pole pieces placed on opposite sides of a head. When an alternating field is switched on, intravenous magnetic particles or magnetic vesicles will be trapped and accumulate along a roughly cylindrical region between the pole pieces. When the field is rotated, the magnetic material will follow. However, due to viscosity and anatomic blood vessel structure, the movement of the magnetic material is hindered. In an extreme case where the field is rotated very quickly, the particles or vesicles will not be able to keep up with the motion. The linear velocity is proportional to the diameter, and thus the field velocity near the center will be much lower, and in fact at the center, it will be zero. Therefore, the peripheral particles or vesicles will be smeared out and eventually distributed at low concentration, whereas the central ones will have a higher concentration. While this does not draw peripheral particles to the center, it creates a concentration difference, thus enabling creation of a higher concentration of particles near the center of rotation, which can be any arbitrary point.

The disclosed magnetic localization schemes can be used to enhance imaging or therapy. For example, contrast agents injected peripherally intravenously are diluted in the blood volume and are also cleared from the blood, so that the concentration at a region of interest is lower than desired, resulting in poor enhancement. By applying an alternating field defined by pole pieces that cover the region to be imaged or by moving the magnetically trapped volume to cover the region of interest, the amount of contrast material can be enhanced many fold. The trapped magnetic material prevents its clearance through the liver or kidneys and the trapping also increases the concentration compared to non-trapped regions. This localization can also be combined with molecular targeting, where the particles or vesicles have a targeting moiety attached, such as an antibody, drug, peptide, or other ligand that binds to a specific target. The magnetic trapping permits a higher concentration of vesicles to interact over time with the target resulting in much higher uptake. The field can then be optionally switched off to allow the material not bound to be released and exit the region, thus leaving the specifically bound material. The unbound vesicles would be diluted in the whole blood volume and their low concentration compared to the targeted region would lead to an enhancement of effect for either imaging or therapy to the desired volume. Alternatively, once a magnetic field is used to trap and concentrate the vesicles in a region and retain them there for an extended period (1 minute to 48 hours), giving the molecular targeting (such as with antibodies) time to be enhanced, the field may be switched off, releasing the unbound vesicles. In this case, however, the released vesicles can be cleared by either waiting for clearance through the kidney, liver, or other organs, or a magneticfield can be placed at another body location away from the treatment volume, or the blood may be extracorporeally shunted and the unbound vesicles removed externally by a magnetic field, thus removing them from the circulation. This design property of removal of excess or unbound vesicles can be crucially important in reducing the toxicity or side effects of the treatment. Since targeting is generally defined as concentration of the material in the desired location compared to concentration in surrounding or other locations, the removal of material not bound in the desired location would greatly enhance targeting.

In the above embodiments, ferromagnetic and other magnetic particles may also be used, since localization with alternating fields is also effective with these particles.

In another embodiment, particles that are ferromagnetic may be further used. As stated earlier, ferromagnets can be problematic due to their residual magnetism causing them to aggregate. However, here we disclose how to use this property to advantage. Ferromagnetic particles can be introduced that have never been magnetized, or have been demagnetized. These will therefore not aggregate. A magnetic field is then applied which will both trap circulating magnetic material and magnetize it. The magnetized material will aggregate in the region the field was applied. The aggregates will then have new properties: their effective size will be larger, their diffusion will be slower, their viscosity may change, and they may be of such size to occlude capillaries or blood vessels. These properties may be used to, for example, enhance imaging and therapy. The aggregates could embolize a tumor, for example. In yet a further embodiment, a rotating static, pulsed or alternating field is applied. At short times, the integrated field is low resulting in low magnetization. Since rotation is slowest at the center of rotation and the particles there are in the field longer, a differential magnetization can be achieved with the most magnetization at the center, thus achieving a defined region of aggregation effect at an arbitrary 3-dimensional position. In this way, deep locations can be magnetically targeted. As before, the magnetic material can be particles carrying additional payloads or the magnetic material can be incorporated into vesicles or cells.

Design of Magnetic Apparatus for Localizing Magnetic Particles and Magnetic Particles in Vesicles

Permanent magnets are generally not ideally suited to in vivo arbitrary 3-dimensional localization of magnetic materials since the materials will be attracted to the closest magnet pole typically outside the body, thus drawing the particles to the skin region. A principle of design is disclosed here to achieve localization at depth. One or more coils are used to produce the field. Pole pieces are used to shape the field such that it is applied across the body or volume in an optimal manner. One such design to achieve this is to run a “C” shaped metal piece or pieces through the center of the coil such that the ends of the “C” fall outside the outer diameter of the coil and form a gap between which the body or volume can be inserted. The “C” design does not have to be a rounded shape, but may be any shape such that the solid part goes through the central region of the coil and the open ends (the “pole pieces”) form a gap between which the subject for localization can be placed. An alternating current is supplied to the coil(s), and the frequency can be 2 Hz to 1 GHz. In initial tests, the convenient 60 Hz was found to be useful. The pole pieces can be shaped to control the localization. The localization of magnetic material will form a similar shaped distribution roughly corresponding to the shape of the opposing pole pieces; i.e., if the pole pieces are thin rectangles, the magnetic material will form a sheet in alignment with the pole pieces. If the pole pieces are pointed, the magnetic material will align roughly along an imaginary line connecting the points of the pole pieces. Use of an alternating field enables the magnetic particles to be distributed throughout the diameter of the volume and not drawn to one side. Other extensions of these designs, or designs that produce distribution of magnetic material at depth will be obvious to those skilled in the art. The localizations described here are when there is an absence of intervening additional constraining aspects of the subject volume, such as internal magnets and morphological barriers. These may modulate the effects described.

Local Permeability Alteration for Better Drug Infusion into Target Tissue

Opening of the vesicles and release of a drug at the desired site is of great value, since other sensitive tissues can be avoided which might cause toxicity if the drugs were applied systemically without targeting. If the vesicles are in the blood stream and target endothelial markers, it may be that release of their contents may not have full effect since the drugs or materials released may be swept away by the blood flow before they can penetrate the target tissue. In addition, the released drug, diagnostic or therapeutic substance may have poor penetration into the tissue due to its size or other properties. To greatly enhance tissue penetration and delivery, it is hereby disclosed to encapsulate not only the drug to be delivered, but a vascular permeability agent, such as vascular endothelial growth factor (VEGF, also known as Vascular Permeability Factor, VPF), C3a, C5a. This factor is able to quickly open endothelial cells such that the flow rate through the blood vessel lining is greatly increased. The drugs or other agents delivered will then have easy access into the target tissue and the effectiveness will be greatly enhanced. For example, a chemotherapeutic drug or antibody therapeutic will now not only be greatly enhanced by being delivered to the tumor site, but will be much more effective because the path through the blood vessel lining of endothelial cells will be opened, permitting the drug to reach the target tumor cells in high concentrations.

For delivery of agents to specific regions of the brain for imaging or therapy, for example to the substantial nigra for treatment of Parkinson's disease, or to tumors, a common problem is the blood brain barrier, that impedes the delivery of drugs, immunological components, and other agents. Here it is disclosed that the vesicle delivery system can not only deliver drugs or agents to a specific brain region, but the vesicles can also contain and release materials that locally disrupt the blood brain barrier (BBB) to allow better penetration of the agents. For example, the vesicles or cells can contain mannitol, RMP-7, activated non-neural specific T cells, or other materials which are known to open the BBB. A previous problem is that mannitol had to be delivered by injection and would affect the whole brain, thus causing excessive toxicity at desired doses. Here, however, the agent to open the BBB can be locally released in higher concentration for better delivery of the therapeutic or imaging agent.

Extracorporeal Removal of Excess Drug Vesicles

Drugs or materials incorporated into the vesicles as described in this invention result in sequestration until released. Since all materials are toxic at some level, the use of biocompatible vesicles permits higher levels to be administered than would be possible for the unencapsulated drug. This is generally true for systemic, subcutaneous, intramuscular, or oral administration. A significant advantage can therefore be obtained in delivering higher concentrations to the site of interest, for example of a cancer therapeutic drug that has high systemic toxicity. However, the loaded vesicles that are not at the target site may release their cargo material in other unwanted tissues that experience some uptake, and this may negate some of the advantage of vesicle delivery. It is here disclosed a method to largely overcome this eventuality by installing an extracorporeal shunt with recognition and removal of the freely circulating vesicles to eliminate any further deposition in unwanted tissues. Extracorporeal shunts are used in dialysis machines for patients with renal insufficiency, where blood is routed to an external filter to remove wastes, then flowed back into the body. In a similar fashion, drug or agent-containing vesicles may be removed. Although this invention uses natural cell membranes, these may be slightly modified before use, when being prepared before injection into the patient. At that time, a recognizable molecule that is non-toxic may be attached to the membrane. This will later be used to remove the excess vesicles. For example, cell membranes can be biotinylated to introduce biotin, which is harmless (biotin is vitamin H). The vesicles are loaded and the imaging or therapy conducted. At an appropriate time, the free vesicles still circulating can be removed by flowing the extracorporeal shunt through an affinity column with immobilized avidin, which tightly binds biotin, and would remove only the modified vesicles.

SubCells

“SubCells” are defined here as a cell-derived vesicles by the methods disclosed herein capable of subdividing cells into one or more smaller membrane-bound vesicles. The ability to easily create many SubCells of various sizes opens up many novel applications. When cells are reformed into smaller vesicles, they will only contain part of the parent cells contents. SubCells without a nucleus will not be able to divide, and are clonogenically sterilized. Tumor cells from a patient can be removed, cloned, and SubCells formed. Sterilized SubCells (those without a nucleus) can be isolated by cell sorting, density centrifugation, or other means. SubCells can then be reinjected to stimulate the immune system without the fear that such cells would form additional tumor growths. Another use of SubCells is in adoptive immunotherapy, where natural killer T cells from a patient are grown ex-vivo to high numbers before reinjection. As mentioned above, a problem is the delivery and concentration of these cells to the tumor. By first forming SubCells, smaller versions of the natural killer T cells are formed that are, for example, one-tenth the normal size. These will have greatly enhanced penetration into tumors. The size of the SubCells may be chosen such that these smaller sized SubCells still retain functional properties of their larger sized parent. SubCells may range in size from just slightly smaller than the parent cell (about 10 microns) down to a small micelle, having a diameter of about 4.5 nm. When SubCells are formed by some disruption to the parent cell membrane, conditions will control not only the final size of the SubCells formed, but the internal contents of the SubCells. If methods are employed that rapidly reseal the disrupted cell membrane, or the cells are packed tightly so that internal contents do not become diluted, little original cellular content will be lost. The SubCells will then retain many of the properties of the parent cells. SubCells can be loaded during the process of their formation according to this disclosure to include new material in their final internal contents. In this way, contrast material, magnetic material, drugs, or other desirable materials can be incorporated into the SubCells if desired.

Another application of SubCells is in wound healing. Because lymphocytes, macrophages, and other cells that are involved in tissue repair must extravesate out of blood vessels to reach the damaged area, creation of functional SubCells of these wound healing involved cells will improve their delivery and ability to extravasate, and healing can be accelerated.

Another application of SubCells is in fighting infections. By creating SubCells of cells involved in bodily defenses, the effectiveness may be improved. For example, many bacteria escape drugs and normal rejection by burrowing deep within muscles and other tissues. Use of SubCells would allow better penetration of immune cells to attack these bacteria, for example.

The disclosed method then provides a novel creation of miniature cells with many of the properties of their parent larger cell. The smaller size will enhance penetration into tumors, wounds, and other tissues. The SubCells can be used to target incorporated agents as well.

Ex Vivo Uses

SubCells provide a more convenient and efficient form of cell material for analysis or binding due to their smaller sizes.

Loaded cells or SubCells can be used to target bone marrow cells, transplant tissues or organs, or cultured cells for studies or directed therapies, such as the destruction of specific cells, such as tumor cells or foreign cells, or delivery of drugs or contrast agents ex-vivo.

SubCells can be small, less than 1 micron, and have good flow, diffusion, and other properties, making them useful in improved lateral flow assays for diseases or conditions, and in improved detection using visible light, infrared, fluorescence, Raman scattering, light and electron microscopy, spectroscopies, and backscattering methods, and other techniques capable of detecting the loaded SubCells

Apparatus to Form SubCells and Load Cells and SubCells

Method 1:

About 5 ml of blood is removed from a patient into a tube with anticoagulant. The tube is put in a robotic “machine” that places the tube in a clinical centrifuge which gently pellets the cell fraction. The machine removes the supernatant by suction, then lowers the suctioned pipette further into the tube to collect the cell pellet. This is mixed with the agent to be incorporated with robotic pipetting. The sample is then withdrawn from the mixing tube and pushed through a filter of the appropriate size. This process can be repeated with the same or other sized filters as required (if better size homogeneity is needed). The filtrate product is then presented at the output station and is ready for use.

An optional stage of processing is after filtration through the membrane, the sample is robotically placed in a container with a large pore dialysis membrane. This container is positioned in a larger container that has biocompatible saline or other desired fluid. The outer container solution may be exchanged with fresh solution if desired. After a predetermined time, the sample will be nearly free of the excess material that was not incorporated, and the loaded cells or cell-derived vesicles may be removed and placed in a product tube ready for use.

Method 2:

An aliquot of blood is removed from a patient into a tube with anticoagulant. The tube is spun in a tabletop clinical centrifuge commonly available in hospitals and laboratories. The serum supernatant is removed and the pelleted cells are mixed with the material to be loaded into the cells or cell-derived vesicles. The material is closely adjusted in osmolarity so as not severely damage or disrupt the cells. The sample is then frozen in liquid nitrogen and thawed in 37° C. water. The freeze-thaw cycle is repeated 2 additional times. This sample can be optionally purified by centrifugation to isolate the loaded vesicles from the excess unincorporated loading material. The sample is then ready for reinjection into the patient.

The above procedure can be automated, where some or all of the manual steps are done robotically by a machine. Blood may be robotically centrifuged followed by automatic withdrawal of supeematant, addition of the material to be loaded, mixing, placing the sample in a cooling environment to freeze it (may be a refrigeration unit or cold solution), removing it for thawing. The sample is then ready for re-injection into the patient. Other additional steps may be similarly handled robotically.

Method 3:

An aliquot of blood is obtained and red cells isolated by centrifugation. Red cells are mixed with the material to be loaded and targeting agent and placed in a mechanical shaker, for example with stainless steel balls, or exposed to sonication. After brief membrane disruption, the sample is ready for patient injection. Other variations include attachment of the targeting moiety after the loading step, or incorporation of targeting agents and use of other membrane loading methods described herein. Some or all of the steps in these procedures may be automated.

Those skilled in the art will realize that various alternatives may be used for the various steps, i.e., those of preparing cells or vesicles, loading them, and purifying them if desired, according to the teachings of this specification and common knowledge. It will also be apparent to those skilled in the art that some or all of the steps may be automated according to the teachings of this specification and common knowledge.

Other Applications

It has been here disclosed a novel delivery system using vesicles, cells, and sub-cells, including mechanisms for targeting such membrane bounded vehicles as well as lysis at a desired region. There are many other applications than described that will be apparent to those skilled in the art. However, a few are specifically disclosed here:

Obesity is a serious problem leading to increased health problems such as diabetes, increased risk of heart disease, back problems, and other ailments including cosmetic ones such as appearance. The targeted drug delivery system herein disclosed may be used to target adipose tissue and release drugs and other effective agents to break down such unwanted adipose tissue, thus providing an effect similar to liposuction but on the molecular scale.

Atherosclerosis can lead to coronary artery disease and stroke. Until now, it has not been possible to safely remove or reduce the arterial plaques except by surgical bypass operations in an emergency. Unfortunately, only some of the patients requiring this are successfully treated, whereas many die before such operations due to plaque rupture and subsequent myocardial infarction or stroke. Here it is disclosed how to target vesicles with therapeutic agents safely and relatively non-invasively to plaque such that it can be treated before such a crisis. For example, cytotoxic or apoptotic inducing agents specific for plaque macrophages or foam cells can be released in order to specifically degrade these offending major components of plaque.

Stroke comes in two forms: blood clots or hemorrhaging. Once the type has been diagnosed (which can be done by the disclosed imaging methods, for example, using a vesicle filled with a contrast agent coated with antibodies to fibrin), the vesicle delivery system disclosed herein can be used to target either clot-buster drugs, such as streptokinase, aspirin, or tissue plasminogen activator (TPA) to dissolve the clot, or agents that can stop hemorrhaging, such as clotting agents. By directly applying these agents in higher doses than can now be safely applied due to the side effects of systemic application, better outcomes can be achieved.

Tumorocidal agents are actually quite effective at killing tumor cells, but doses are limited by systemic side effects. Using the disclosed drug delivery system of targeted vesicles and cells, chemotherapy agents, such as taxol, cis-platin, alkylating agents, antibodies, methotrexate and others can be safely applied regionally tumors in higher concentrations for more effective results.

### EXAMPLES

### Example 1

### Loading Red Blood Cell (RBC) Vesicles with the Dye Trypan Blue

Human blood was drawn into heparinized tubes. One milliliter (ml) was mixed with 9 ml of phosphate buffered saline, pH 7.4, with a molarity of 0.15 M, and spun for five minutes at 1,000×g to wash and pellet the red cells; the supernatant was removed and discarded. 0.1 ml of an isotonic 0.4% trypan blue (a highly colored blue dye) solution was added to 0.1 ml of the packed red cells. The cells were then filtered through a 3 micron filter two times. Vesicles were purified by centrifugation. Microscopic observation revealed many small vesicles, all less than 3.5 microns, and many less than 0.5 microns. Vesicles appeared intensely colored, indicating loading with the dye.

### Example 2

### Loading Red Blood Cell (RBC) Vesicles with Gadolinium (Gd)

Human blood was drawn into EDTA phlebotomy tubes. Four milliliters (ml) was mixed with 10 ml of 5 mM phosphate buffer, pH 7.4, containing 75 mM NaCl, and spun for three minutes at 2,000 rpm in a swinging bucket centrifuge to wash and pellet the red cells. The supernatant was removed and discarded. 0.7 ml of gadodiamide (0.5 M, Omniscan®) was mixed with 1.66 ml of the pellet, producing an average molarity of about 0.2 M. The cells were then filtered through a 5 micron, then a 3 micron filter. Microscopic observation revealed many small vesicles, most less than 3.5 microns, and many less than 0.5 microns. The values used for the ionic strength of the various components was done so as to maximize loading and to produce a final molarity so as to maintain vesicle integrity when intravenously injected.

### Example 3

### MRI Imaging with Gadolinium Loaded Cell Vesicles, Demonstrating Vascular Imaging, Blood Pool Imaging, and Improved Tumor Detection

A male rat bearing a subcutaneous F98 glioma tumor in its thigh was anesthetized and a catheter inserted into the femoral vein. The animal was then placed in a 1.5 Tesla clinical MRI scanner with a head coil around it. T1 images were acquired before injection. The sample in example 2 was used without further purification, and an amount was injected, corresponding to a dose of 0.1 mmol Gd/kg, which is the recommended dose/weight for gadodiamide use in vivo. Images were acquired using both T1 and T2 modes. The first images minutes after injection and those collected up to 20 minutes or more later showed very high vascular contrast in the T1 mode. At 10 minutes post injection the abdominal aorta, the inferior vena cava, the hepatic portal vein, the vasculature of the liver, and the tumor were clearly contrasted compared to the image taken before the injection. For comparison, a rat bearing a similar tumor was injected with 0.1 mmol gadodiamide/kg. That rat showed a maximal tumor contrast approximately one-half the intensity of the rat given the vesicle-loaded gadodiamide, but at all times assayed the vessels were not significantly contrasted. Other tissues, such as the lungs showed more contrast in the vesicle-loaded preparation. By 45 minutes, the contrast in the liver had virtually cleared, indicating the vesicles were not being trapped by the liver. Many of the smaller vesicles filtered through the kidneys, since at 30 min post injection not only was contrast seen in the urine in the bladder (as also seen with the gadodiamide only preparation), but high contrast was seen on the surface of the urine in the bladder. This may be explained by the lower density of the lipid-containing vesicles, allowing them to float on the surface. No toxicity was observed in the animal receiving the red-cell derived vesicles. It should be noted that whatever the fate of the contrast agent is, only an FDA approved standard amount was injected, and should not cause any toxic effects.

Quantitatively, 10 minutes after the red cell vesicles loaded with Gd was injected, the heart T1 contrast increased from 241±138 before injection to 1032±206 Hounsfield units (HU), the liver increased from 782±29 to 1019±27 HU, the abdominal aorta increased from 637±80 to 1823±92 HU, the inferior vena cava increased from 600±106 to 1509±68, the hepatic portal vein increased from 601±55 to 1580±250, the tumor increased from 421±65 to 1398±49 HU, the brain increased from 508±15 to 700±26 HU, the kidney increased from 667±86 to 1443±106.

By comparison, 10 minutes after injection of 0.1 mmol/kg gadodiamide, the heart contrast changed from 449±123 HU before injection to 540±137 HU after injection, the liver changed from 789±32 to 804±58 HU, the kidney increased from 563±36 to 1411±136, the abdominal aorta changed from 618±42 to 627±76, the inferior vena cava changed from 760±56 to 770±49, the hepatic portal vein changed from 678±122 to 774±47, the liver changed from 815±16 to 831±27, and the tumor changed from 507±15 to 828±82 HU. From these data it is apparent that the new contrast agent and methods produces significantly better contrast in virtually all organs, and is an excellent blood pool agent (Table 1).

TABLE 1
Change in contrast 10 min. after injection of gadodiamide or gadolinium
filled red cell vesicles. The Factor of Improvement is the ratio of
the percent change in contrast for the vesicle preparation compared
to the percent change in contrast for the standard gadodiamide.

| | | Before | After | Contrast | | |
|---|---|---|---|---|---|---|
| | | injection | injection | change | Percent | Factor of |
| tissue | | (HU) | (HU) | (HU) | change | improvement |
| Heart | Gd-vesicles | 241 | 1032 | 791 | 328 | 16.4 |
| | gadodiamide | 449 | 540 | 91 | 20 | |
| Liver | Gd-vesicles | 754 | 967 | 213 | 28 | 14.0 |
| | gadodiamide | 815 | 831 | 16 | 2 | |
| Kidney | Gd-vesicles | 667 | 1443 | 776 | 116 | 0.8 |
| | gadodiamide | 563 | 1411 | 848 | 151 | |
| Abdominal | Gd-vesicles | 637 | 1823 | 1186 | 186 | 186.0 |
| Aorta | gadodiamide | 618 | 627 | 9 | 1 | |
| Inferior | Gd-vesicles | 600 | 1509 | 909 | 152 | 151.0 |
| vena cava | gadodiamide | 760 | 770 | 10 | 1 | |
| hepatic | Gd-vesicles | 601 | 1580 | 979 | 163 | 11.6 |
| portal vein | gadodiamide | 678 | 774 | 96 | 14 | |
| tumor | Gd-vesicles | 421 | 1398 | 977 | 232 | 3.7 |
| | gadodiamide | 507 | 828 | 321 | 63 | |

### Example 4

### Loading Red Blood Cell Vesicles with Dye by Freeze-Thawing

Human blood was drawn into heparinized tubes. One milliliter (ml) was mixed with 9 ml of phosphate buffered saline, pH 7.4, with a molarity of 0.15 M, and spun for five minutes at 1,000×g to wash and pellet the red cells; the supernatant was removed and discarded. 0.1 ml of an isotonic 0.4% trypan blue (a highly colored blue dye) solution was added to 0.1 ml of the packed red cells. The cells were then frozen either by immersing a tube into liquid nitrogen, placing a tube in a freezer at −20 deg. C., or placing a tube in a freezer at −80 deg. C. Samples were then thawed. Microscopic observation revealed many small vesicles, all less than 5 microns, and many less than 0.5 microns. Vesicles appeared intensely colored, indicating loading with the dye.

### Example 5

### Loading Red Blood Cell Vesicles with Dye by Sonication

Human blood was drawn into heparinized tubes. One milliliter (ml) was mixed with 9 ml of phosphate buffered saline, pH 7.4, with a molarity of 0.15 M, and spun for five minutes at 1,000×g to wash and pellet the red cells; the supernatant was removed and discarded. 0.1 ml of an isotonic 0.4% trypan blue (a highly colored blue dye) solution was added to 0.1 ml of the packed red cells. The cells were then sonicated with a 100 watt microtip sonicator (Misonix) for 5 sec at power setting 10. Microscopic observation revealed many small vesicles, all less than 5 microns, and many less than 1 micron. Vesicles appeared intensely colored, indicating loading with the dye.

### Example 4

### Loading Red Blood Cell Vesicles with Dye by Mechanical Disruption

Human blood was drawn into heparinized tubes. One milliliter (ml) was mixed with 9 ml of phosphate buffered saline, pH 7.4, with a molarity of 0.15 M, and spun for five minutes at 1,000×g to wash and pellet the red cells; the supernatant was removed and discarded. 0.1 ml of an isotonic 0.4% trypan blue (a highly colored blue dye) solution was added to 0.1 ml of the packed red cells. The cell suspension was then loaded into a stainless steel vessel with three 9 mm stainless steel balls and placed in a shaker device and shaken for 40 sec. Microscopic observation revealed many small vesicles, most less than 5 microns. Vesicles appeared blue colored, indicating loading with the dye. Samples retained their color upon storage for at least several days.

### Example 5

### Loading Red Blood Cell Vesicles with Gold Nanoparticles

Human blood was drawn into heparinized tubes. One milliliter (ml) was mixed with 9 ml of phosphate buffered saline, pH 7.4, with a molarity of 0.15 M, and spun for five minutes at 1,000×g to wash and pellet the red cells; the supernatant was removed and discarded. 0.1 ml of a gold nanoparticle solution (˜2nm particles suspended in phosphate buffered saline, pH 7.4) was added to 0.1 ml of the packed red cells. The cell suspension was then loaded into a stainless steel vessel with three 9 mm stainless steel balls and placed in a shaker device and shaken for 40 sec. Microscopic observation revealed many small vesicles, most less than 5 microns. Vesicles appeared brown colored, indicating loading with the gold nanoparticles.

### Example 6

### Increasing the Loaded Vesicle Size by Heating

The gold nanoparticle vesicles prepared in example 5 were heated to 100 degrees C for 1, 2, 3, or 4 minutes. Four minutes of heating caused the red cell vesicles to fuse and form larger vesicles, some 5 microns in size, and also tubes and joined vesicle structures, some linear or branched. The single vesicles and other coalesced vesicle structures retained their initial high loading of gold nanoparticles and appeared brown colored in their interior. Three minutes of heating also caused fusion of small vesicles to form larger ones, with fewer larger fused aggregates. Heating for 2 minutes produced mostly large single vesicles 2 to 5 microns in size, with few larger aggregates. Heating for 1 minute had a lesser effect.

### Example 7

### CT Imaging with Gold Nanoparticle Loaded Vesicles Heated to Produce Larger Vesicles

The red cell vesicles were loaded with gold nanoparticles as in example 5, then heated to 100 deg. C. for 2 min. as per example 6, then injected intravenously into a mouse via tail vein injection. The animal was anesthetized and placed in a Skyscan microCT unit and imaged. Blood vessels were clearly seen 20 min post injection, and little uptake of the contrast agent was seen in the kidney or liver, whereas the gold nanoparticles by themselves when injected were cleared through the kidney, noticeable shortly after injection.

### Example 8

### Loading Red Blood Cell Vesicles with Iodine

Because the cells and cell-derived vesicles are impermeable to water soluble materials, they may be loaded with other materials. To test this, red cells were loaded with iodine contrast medium.

Human blood was drawn into heparinized tubes. One milliliter (ml) was mixed with 9 ml of phosphate buffered saline, pH 7.4, with a molarity of 0.15 M, and spun for five minutes at 1,000×g to wash and pellet the red cells; the supernatant was removed and discarded. 0.1 ml of a 0.15 M solution of iodine contrast medium (iohexol) was added to 0.1 ml of the packed red cells. The cell suspension was then loaded into a stainless steel vessel with three 9 mm stainless steel balls and placed in a shaker device and shaken for 40 sec. Microscopic observation revealed many small vesicles, most less than 5 microns.

### Example 9

### Immunologic Targeting of Loaded Red Blood Cell Vesicles

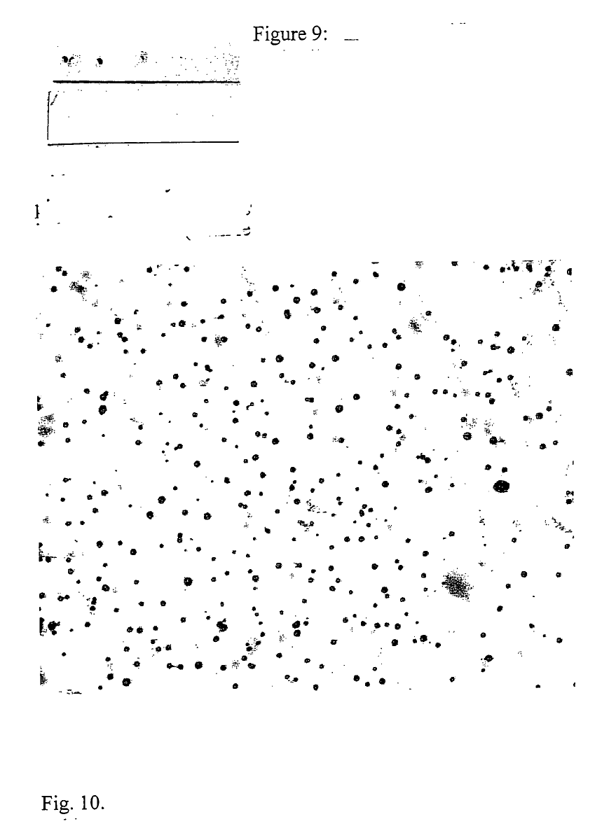

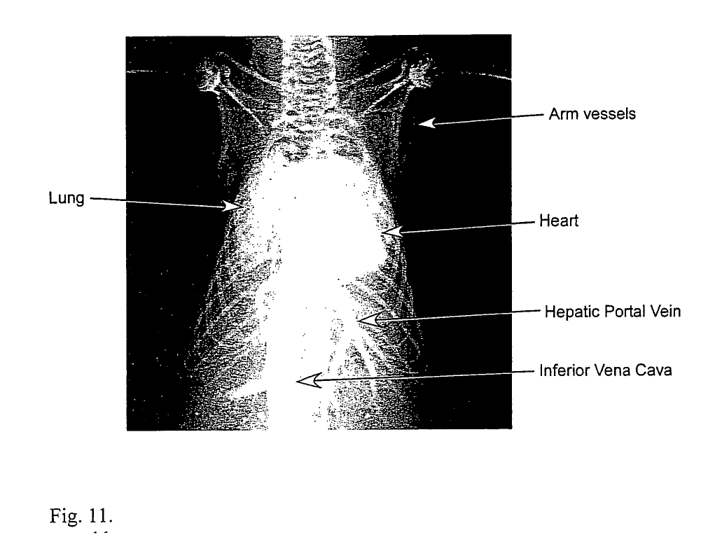

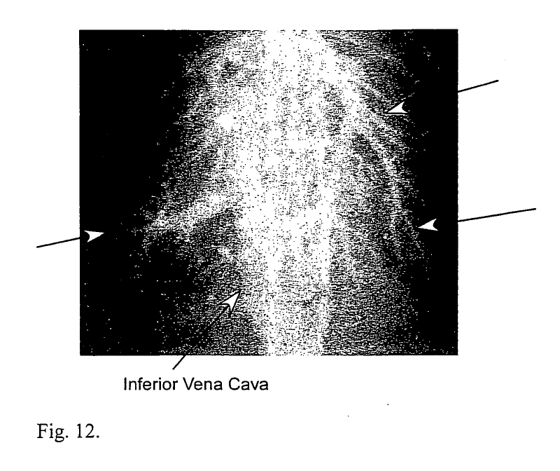

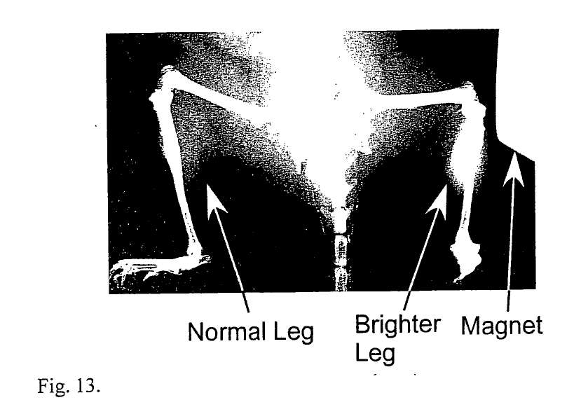

A further advantage of these red cell vesicles is that antibodies, antibody fragments, or peptides may be easily covalently linked to them. Experiments were done to validate this. Red cells were reacted with sulfosuccinimidyl 4-[p-maleimidophenyl] butyrate to convert some amino groups to maleimide. Goat anti-mouse F(ab′)₂ was reduced with mercaptoethylamine and purified on a desalting column. The two solutions were mixed and incubated. Removal of excess antibody was achieved by centrifuging the red cells. To demonstrate specific immunoreactivity, dilutions of mouse IgG were made on nitrocellulose paper (see FIG. 9). The conjugated red cells were then incubated for 35 min. and washed. Targeting was evident from the red hemoglobin color at the target spots. An identical concentration of red cells that had not been coupled to antibody was incubated with the target panel and showed no binding.

### Example 10

### CT Imaging of Red Blood Cell Vesicles Loaded with Iodine

Red blood cell vesicles were loaded with iodine contrast medium as described in example 8. These were then injected intravenously into mice via the tail vein and X-ray imaging performed with a Skyscan MicroCT unit. Arteries and veins could be clearly seen 10 minutes and longer after injection, and loss of iodine to the extravascular space and kidneys as rapidly occurs with the iodine contrast medium itself, was greatly reduced, permitting blood pool imaging.

### Example 11

### Loading Full-Sized Red Cell Membranes and Smaller Vesicleswith Gold Nanoparticles

Whole blood was collected in EDTA to prevent clotting. Cells were washed in 5 mM sodium phoshphate buffer pH 8 containing 150 mM sodium chloride by centrifuging the cells at 2.2 krpm for 4 minutes and discarding the supernatant, along with the “buffy coat”, or top layer of the pellet that contains other cells. This operation was done twice. The cells were then hypotonically lysed by adding an a 40-fold volume excess of ice cold 5 mM phosphate buffer, pH 8 and mixing by tube inversion. Cell membranes were then isolated in concentrated form by centrifugation in a SS34 rotor at 15,000 rpm (about 20 kg) for 20 minutes. The supernatant was discarded as well as the hard part of the pellet that contained other cell types and unlysed cells. This operation was done only once. An equal volume of gold nanoparticles, 1.9 nm in diameter at a concentration of 270 mg Au/ml, suspended in water, was added to the purified membranes and incubated with them on ice for 30 minutes. The mixture was then adjusted to 150 mM in salt, by adding a concentrated buffer solution, 100 mM phosphate, pH8, containing 3 M sodium chloride, so that the final concentration was 150 mM sodium chloride. The mixture was then incubated at 37 degrees C. for 30 minutes. The latter operations result in sealing of the cells and vesicles. Loading in this way resulted in many normally sized cell membranes, while some smaller loaded vesicles were also formed. These vesicles could be purified by centrifugation to separate them from unencapsulated gold nanoparticles. The sealed membranes retained the gold nanoparticles for at least several days.

### Example 12

### Preparation of Small Vesicles Loaded with Gold Nanoparticles from Red Blood Cell Membranes by a Heating Step

Whole blood was collected in EDTA to prevent clotting. Cells were washed in 5 mM sodium phoshphate buffer pH 8 containing 150 mM sodium chloride by centrifuging the cells at 2.2 krpm for 4 minutes and discarding the supernatant, along with the “buffy coat”, or top layer of the pellet that contains other cells. This operation was done twice. The cells were then hypotonically lysed by adding an a 40-fold volume excess of ice cold 5 mM phosphate buffer, pH 8 and mixing by tube inversion. Cell membranes were then isolated in concentrated form by centrifugation in a SS34 rotor at 15,000 rpm (about 20 kg) for 20 minutes. The supernatant was discarded as well as the hard part of the pellet that contained other cell types and unlysed cells. This operation was done only once. An equal volume of gold nanoparticles, 1.9 nm in diameter at a concentration of 270 mg Au/ml, suspended in water, was added to the purified membranes and incubated with them on ice for 30 minutes. The mixture was then adjusted to 150 mM in salt, by adding a concentrated buffer solution, 100 mM phosphate, pH8, containing 3 M sodium chloride, so that the final concentration was 150 mM sodium chloride. The mixture wais then incubated at 37 degrees C. for 30 minutes. The latter operations result in sealing of the cells and vesicles. Loading in this way resulted in many normally sized cell membranes, while some smaller loaded vesicles were also formed. These vesicles could be purified by centrifugation to separate them from unencapsulated gold nanoparticles. The sealed membranes retained the gold nanoparticles for at least several days.

### Example 13

### X-ray Imaging of Vesicles from Red Blood Cells Loaded with Gold Nanoparticles

The vesicles of Example 5 were injected intravenously by tail vein into mice and imaged with a clinical mammography unit (Lorad Medical Systems model XDA101827) operating at 22 kVp. Blood vessels and vascular trees were seen with unusual clarity and resolution.

### Example 14

### Loading Vesicles from Red Blood Cells with Magnetic Nanoparticles and Demonstration of Magnetic Properties

Whole blood was washed two times with 5 mM phosphate buffer, 150 mM sodium chloride, pH 8 by dilution of 1 ml into 8 ml of buffer and centrifugation at 2.2 krpm for 4 min in an IEC tabletop centrifuge. Washed cells were then converted to ghosts by dilution 1:30 in cold 5 mM phosphate buffer, pH 8. After inversion, ghosts were isolated by centrifugation for 30 min at 15 krpm in a SS34 rotor in a RC5B centrifuge. 20 microliters of ghosts were mixed with 20 microliters of anionic, water soluble, 10 nm iron superparamagetic nanoparticles. The sample was then frozen and thawed twice using liquid nitrogen. 20 times concentrated 5 mM phosphate buffer, 150 mM sodium chloride, pH 5.5 was added to adjust the salt concentration to approximately 150 mM. The preparation was warmed to 60° C. for 1 minute. Dilution into 10 mM phosphate buffer, 150 mM sodium chloride, pH 7.4 (PBS) and observation by light microscopy revealed many 0.2-5 micron vesicles with a brown color, the color of the ferrofluid.

The ferrofluid itself with the same concentration as in the vesicle preparation, which was colored, was held against a magnet pole (˜10,000 gauss) and showed no visible attraction to it, even after several minutes. Surprisingly, when the magnetic vesicles were similarly placed, all of the colored solution quickly accumulated near the pole and the solution became clear after only a few seconds.

### Example 15

### Loading Vesicles from Red Blood Cells with Magnetic and Gold Nanoparticles and Demonstration of In Vivo Image Enhancement

Whole blood was washed two times with 5 mM phosphate buffer, 150 mM sodium chloride, pH 8 by dilution of 1 ml into 8 ml of buffer and centrifugation at 2.2 krpm for 4 min in an IEC tabletop centrifuge. Washed cells were then converted to ghosts by dilution 1:30 in cold 5 mM phosphate buffer, pH 8. After inversion, ghosts were isolated by centrifugation for 30 min at 15 krpm in a SS34 rotor in a RC5B centrifuge. 30 microliters of ghosts were mixed with 30 microliters of anionic, water soluble, 10 nm iron superparamagetic nanoparticles, and 30 microliters of 1.9 nm gold nanoparticles having a gold concentration of 0.6 g/ml. The sample was then frozen and thawed twice using liquid nitrogen. 20 times concentrated 5 mM phosphate buffer, 150 mM sodium chloride, pH 5.5 was added to adjust the salt concentration to approximately 150 mM. The preparation was warmed to 60° C. for 1 minute. Dilution into 10 mM phosphate buffer, 150 mM sodium chloride, pH 7.4 (PBS) and observation by light microscopy revealed many 0.2-5 micron vesicles with a brown color. The magnetic vesicles were purified by placing the sample tube near a permanent magnet (˜10,000 gauss) and removing the adjacent fluid, with repeated washes of PBS.

The preparation in 0.2 ml PBS was injected intravenously into a 20 g mouse by tail vein and the leg held near a magnet pole. X-ray imaging revealed a high contrast due to the gold in the leg near the magnet.

### Example 16

### Localization of Magnetic Vesicles and Magnetic Materials by Alternating Fields

Coils were constructed using 800 turns of 20 ga magnet wire with an inside diameter of 21 mm. Pole pieces were cut from a steel plate 1 mm thick. Several designs were tested: one was similar to a horseshoe (or “C” shape) where it was threaded through the inner hole of the coil and the open ends protruded past the outer diameter of the coil. The tips of the open ends were cut to approach each other to form pole pieces with a gap where the flux would travel across. The gap was 13 mm. In one case each pole piece had a width of 11 mm and in another design the pole pieces were pointed. A test tube containing either iron filings in water, red blood cell vesicles loaded with ferrofluid 10 nm particles (example 14), or red blood cell vesicles loaded with ferrofluid 10 nm particles and 1.9 nm gold nanoparticles (example 15) in buffer was inserted between the pole pieces.

5 amperes of 60 Hz alternating current was supplied from a transformer with a 10 ohm resistor in series and a 250 microfarad capacitor in parallel to the coil. All of the magnetic materials behaved similarly. With the 11×1 mm pole pieces, the magnetic material in the aqueous tube lined up as a sheet across the full width of the tube in the same orientation of the pole piece with a maximum width of 1.5 mm. With the pointed pole pieces, the material lined up across the full width of the tube in a column approximately 1 mm in diameter.

### Example 17

### Biodistribution of Red Cell Derived Vesicles Loaded with Gold Nanoparticles

Red blood cell ghosts were prepared as described in example 14. 200 microliters of packed ghosts were dried to 100 microliters by pumping during centrifugation using a Speedvac device. 50 microliters of 300 mg Au/ml 1.9 nm gold nanoparticles were dried using the same device. The two components were mixed and the solution frozen in liquid nitrogen and thawed twice. The vesicles were then adjusted to approximately 150 mM salt by adding a 20-fold concentrated buffer containing 3 M NaCl, 100 mM phosphate buffer, pH 5.5. The preparation was heated for 1 minute at 60° C. Observation by light microscopy after dilution into PBS revealed many 0.2-8 micron sized vesicles that were brown in color indicating gold incorporation. The vesicles were purified from their external solution by filtration of a 0.1 micron filter where the retentate was retained. Three washes with PBS were used and the retentate showed a high concentration of loaded vesicles. The preparation was filtered through a 5 micron filter and injected into the tail vein of a mouse bearing a squamous cell carcinoma, SCCVII implanted subcutaneously in its thigh. After 4 minutes, the animal was killed by CO₂ inhalation and samples of blood, tumor, normal muscle, liver and kidney were removed and placed in tared vials. The samples were then dissolved in nitric acid and aqua regia and the gold content analyzed by graphite furnace atomic absorption spectrometry. Gold analysis showed that the concentration in the injectate was 7.59±0.27 mg Au/ml. 0.3 ml was injected, giving an injected dose of 2.28±0.08 mg Au. Tissue analysis revealed the distribution shown in Table 2. This distribution was compared with an injection of the free 1.9 nm gold nanoparticles. Notably, approximately twice remained in the blood when the gold was in the vesicles at this time point, indicating that imaging and blood delivery would be enhanced. As may be expected from a larger material, liver localization increased, whereas kidney levels were decreased compared to the free gold nanoparticles. Muscle levels were only 60% of what they were for the free gold particles, whereas tumor levels were approximately the same. Important in specific delivery, the tumor to non-tumor ratio (here tumor-to-muscle), was therefore increased by using the vesicles by a factor of 1.75, a 75% significant increase.

TABLE 2
Biodistribution of gold after injection of gold nanoparticle-loaded
red blood cell ghost membranes four minutes after injection
intravenously into a mouse. % ID/g = % injected dose per
gram of tissue, SD = standard deviation.

| | Vesicles containing | | 1.9 nm gold | |
|---|---|---|---|---|
| | 1.9 nm nanoparticles | | nanoparticles | |
| | % ID/g | SD | % ID/g | SD |
| Blood | 39.9% | 2.3 | 20.1% | 0.7 |
| Liver | 16.9% | 1.0 | 5.8% | 0.2 |
| Kidney | 20.6% | 1.1 | 30.3% | 0.9 |
| Muscle | 1.2% | 0.1 | 2.0% | 0.1 |
| Tumor 1 | 2.8% | 0.1 | 2.7% | 0.4 |
| Tumor 2 | 2.8% | 0.2 | 2.5% | 0.1 |
| Tumor1/Muscle Ratio | 2.33 | 0.22 | 1.37 | 0.45 |
| Tumor2/Muscle Ratio | 2.30 | 0.19 | 1.27 | 0.09 |

### Example 18

### Lysis of Vesicles to Deliver a Drug

In this hypothetical example, a sample of a patient's blood is removed by phlebotomy. The blood was washed two times with 5 mM phosphate buffer, 150 mM soldium chloride, pH 8 by dilution of 1 ml into 8 ml of buffer and centrifugation at 2.2 krpm for 4 min in an IEC tabletop centriguge. Washed cells were then converted to ghosts by dilution 1:30 in cold 5 mM phosphate buffer, pH 8. After inversion, ghosts were isolated by centrifugation for 30 min at 15 krpm in a SS34 rotor in a RC5B centrifuge. Red blood cell ghosts were mixed with an equal volume of water soluble, iron superparamagetic nanoparticles also containing 1.5 mg/ml cisplatin. The sample was then frozen and thawed twice using liquid nitrogen. 20 times concentrated 5 mM phosphate buffer, 150 mM sodium chloride, pH 5.5 was added to adjust the salt concentration to approximately 150 mM. The preparation was warmed to 60° C. for 1 minute. Dilution into 10 mM phosphate buffer, 150 mM sodium chloride, pH 7.4 (PBS) and observation by light microscopy revealed many 0.2-5 micron vesicles with a brown color, the color of the ferrofluid. The vesicles showed strong attraction to a magnetic pole, which was then used for further purification. DNA fragments were then coupled to the outer surface of the vesicles using a covalent crosslinker. The vesicles were purified from excess reagents by magnetic separation and injected intravenously into the patient. A magnetic field was then used to localize the vesicles to a tumor region. A second intravenous injection was then given of a humanized anti-DNA antibody. This antibody circulated and bound to the loaded red cell ghosts being held in the tumor region by the magnetic field. Once bound, the anti-DNA antibodies triggered complement lysis of the vesicles releasing the anti-cancer drug cisplatin. It was found that a more than 10-fold increase in concentration of the drug could be thusly delivered to the tumor than by normal systemic drug infusion without increasing harmful toxic reactions in the rest of the body. An improved tumor response was achieved.

What is claimed is:

1. A method of forming loaded cells, cell-derived vesicles or synthetic vesicles for facilitating in vivo imaging, targeting or biological modification of tissue, which comprises loading cells or cell-derived vesicles by mechanically shaking said cells or vesicles in the presence of an active substance intended to facilitate imaging, targeting, or biological modification, such active substance loaded cells upon injection into a host having a sufficiently long active life before disintegration or removal in the host to enable the intended imaging, targeting, or biological modification.

2. The method as claimed in claim 1 wherein the mechanical shaking is effected with one or more hard objects.

3. The method as claimed in claim 2 wherein the hard objects are metal balls, glass balls, ceramic balls, plastic balls or teflon balls.

4. The method as claimed in claim 1 wherein the shaking is effected in a metal container.

5. The method as claimed in claim 3 wherein the shaking is effected for 10 seconds to 10 minutes.

6. The method as claimed in claim 1 wherein the frequency of oscillation of shaking is between 1 and 60 cycles per second.

7. The method as claimed in claim 1 where the active substance to facilitate imaging is chosen from the group of agents containing Gd, Dy, Mn, Co, Ni, Fe, I, Au, W, In, Tc, C, F, Bi, and Tl.

8. The method as claimed in claim 1 where the active substance to facilitate targeting is chosen from the group of agents containing a drug, a protein, a peptide, an antibody, an antibody fragment, a ligand, a cytokine.

9. The method as claimed in claim 1 where the active substance to facilitate biological modification is chosen from the group of agents containing a drug, a protein, a peptide, an antibody, an antibody fragment, a ligand, a cytokine, an inhibitory substance, a stimulatory substance.

10. A method of forming loaded cells, cell-derived vesicles, or synthetic vesicles for facilitating in vivo imaging, targeting, or biological modification of tissue, which comprises loading cells or cell derived vesicles by first freezing and then thawing said cells or vesicles in the presence of an active substance intended to facilitate imaging, targeting or biological modification, such active substance loaded cells or vesicles upon injection into a host having a sufficient long active life before disintegration or removal in the host to enable the intended imaging, targeting, or biological modification.

11. The method as claimed in claim 10 where the active substance to facilitate imaging is chosen from the group of agents containing Gd, Dy, Mn, Co, Ni, Fe, I, Au, W, In, Tc, C, F, Bi, and Tl.

12. The method as claimed in claim 10 where the active substance to facilitate targeting is chosen from the group of agents containing a drug, a protein, a peptide, an antibody, an antibody fragment, a ligand, a cytokine.

13. The method as claimed in claim 10 where the active substance to facilitate biological modification is chosen from the group of agents containing a drug, a protein, a peptide, an antibody, an antibody fragment, a ligand, a cytokine, an inhibitory substance, a stimulatory substance.

14. The method as claimed in claim 10 wherein the freezing rate is between 0.1 second to 5 minutes

15. The method as claimed in claim 10 wherein the freeze-thawing procedure is repeated 1 to 5 times.

16. A method of forming loaded cells, cell-derived vesicles, or synthetic vesicles for facilitating in vivo imaging, targeting or biological modification of tissue, which comprises loading cells or cell derived vesicles by passing said cells or vesicles through a porous material in the presence of an active substance intended to facilitate imaging, targeting, or biological modification such active substance loaded cells upon injection into a host having a sufficiently long active life before disintegration or removal in the host to enable the intended imaging, targeting, or biological modification.

17. The method as claimed in claim 16 where the active substance to facilitate imaging is chosen from the group of agents containing Gd, Dy, Mn, Co, Ni, Fe, I, Au, W, In, Tc, C, F, Bi, and Tl.

18. The method as claimed in claim 16 where the active substance to facilitate targeting is chosen from the group of agents containing a drug, a protein, a peptide, an antibody, an antibody fragment, a ligand, a cytokine.

19. The method as claimed in claim 16 where the active substance to facilitate biological modification is chosen from the group of agents containing a drug, a protein, a peptide, an antibody, an antibody fragment, a ligand, a cytokine, an inhibitory substance, a stimulatory substance.

20. The method of claim 16 where the porous material is a membrane with effective pore sizes selected from the range 0.02 to 8 microns.

21. The method of claim 16 or 20 wherein the passing through the porous material is repeated 1 to 10 times.

22. A method of forming loaded cells, cell-derived vesicles, or synthetic vesicles for facilitating in vivo imaging, targeting or biological modification of tissue, which comprises loading cells or cell derived vesicles by fusing said cells or vesicles with liposomes or vesicles containing an active substance intended to facilitate imaging, targeting, or biological modification such active substance loaded cells upon injection into a host having a sufficiently long active life before disintegration or removal in the host to enable the intended imaging, targeting, or biological modification.

23. The method as claimed in claim 22 where the active substance to facilitate imaging is chosen from the group of agents containing Gd, Dy, Mn, Co, Ni, Fe, I, Au, W, In, Tc, C, F, Bi, and Tl.

24. The method as claimed in claim 22 where the active substance to facilitate targeting is chosen from the group of agents containing a drug, a protein, a peptide, an antibody, an antibody fragment, a ligand, a cytokine.

25. The method as claimed in claim 23 where the active substance to facilitate biological modification is chosen from the group of agents containing a drug, a protein, a peptide, an antibody, an antibody fragment, a ligand, a cytokine, an inhibitory substance, a stimulatory substance.

26. A method of enlarging the size of cells, cell-derived vesicles, or synthetic vesicles optionally loaded with an active substance intended to enable imaging, targeting, or biological modification by fusing two or more cells, cell-derived vesicles, or synthetic vesicles.

27. The method as claimed in claim 26 where the active substance to facilitate imaging is chosen from the group of agents containing Gd, Dy, Mn, Co, Ni, Fe, I, Au, W, In, Tc, C, F, Bi, and Tl.

28. The method as claimed in claim 26 where the active substance to facilitate targeting is chosen from the group of agents containing a drug, a protein, a peptide, an antibody, an antibody fragment, a ligand, a cytokine.

29. The method as claimed in claim 26 where the active substance to facilitate biological modification is chosen from the group of agents containing a drug, a protein, a peptide, an antibody, an antibody fragment, a ligand, a cytokine, an inhibitory substance, a stimulatory substance.

30. The method of claim 26 wherein heating is used to induce the membrane fusion.

31. The method of claim 30 wherein the heating is in the range of 35to 100° C.

32. The method of claim 26 wherein chemicals are used to induce the membrane fusion, said chemicals being selected from the group consisting of polyethylene glycol and calcium phosphate.

33. The method of claim 1 wherein the loaded cells or vesicles and free active substance are injected into an animal whereby a dual probe with properties of both encapsulated and free substance are obtained.

34. A method as claimed in claim 1 wherein the active substance is at least one image enhancing contrast agent.

35. The method of claim 34 wherein said imaging enhancing contrast agent is chosen from the group of agents containing Gd, Dy, Mn, Co, Ni, Fe, I, Au, W, In, Tc, C, F, Bi, and Tl.

36. The method of claim 34 wherein said imaging enhancing contrast agent is chosen from the group consisting of: iodine contrast agents, gadolinium contrast agents, dysprosium contrast agents, manganese contrast agents, radioactive contrast agents, positron emission tomography contrast agents and gold nanoparticles.

37. The method of claim 34 wherein said imaging enhancing contrast agent is chosen from the group comprising: molecules or particles useful for fluorescent detection including fluorophores, quantum dots, and phosphors; molecules or particles useful for Raman scattering and spectroscopy including organic molecules and metal particles.

38. The method of claim 34 wherein said imaging enhancing contrast agent is useful for imaging by MRI, X-ray, PET, SPECT, fluorescence, and Raman scattering.

39. A method as claimed in claim 34 wherein the active substance is a target specific drug.

40. A method as claimed in claim 1 wherein the cells or cell derived vesicles are red blood cells.

41. A method as claimed in claim 1 wherein the loaded cells are heated prior to use to enlarge their size.

42. A method as claimed in claim 1 wherein the cells or cell derived vesicles are formed from red blood cells of the host to be imaged or targeted.

43. A method of facilitating CT, planar X-ray, or MRI imaging which comprises withdrawing blood from the host to be subjected to the X-ray and MRI imaging, loading the withdrawn blood with a contrast agent and reinjecting the product as obtained into said host, said contrast agent having a sufficiently long active life to enhance and perform the imaging before disintegration.

44. A method as claimed in claim 43 wherein the loaded blood cells are heated prior to use to expand them.

45. The loaded cell product obtained by the method of claims 1, 2, 10, 14, 16, 22, 34 or 35.

46. A method as claimed in claim 1 for use in targeting specific sites in the host, wherein surface binding moieties are attached to the loaded cells; said moieties being selected from the group consisting of proteins, antibodies, antibody fragments, peptides, drugs, and molecules with binding affinity to the desired target.

47. The loaded cell product obtained by the method of claim 46.

48. A method as claimed in claim 1 wherein red blood cell vesicles are loaded with active substance, said method comprising: drawing human blood into a receptacle, washing the red blood cells, spinning the product thus obtained, removing the supernatant liquid and mixing the packed red blood cells thus obtained with an active substance to obtain a cell suspension, shaking the cell suspension in a receptacle under mechanical stress to obtain small vesicles of a size of less than five microns which retain the encapsulated active substance upon storage for several days.

49. The method of claim 48 wherein the blood is washed with phosphate buffered saline solution of a pH of about 7.4.

50. A method as claimed in claim 48 wherein the concentration of the encapsulated active substance is between 20 and 300 mM.

51. A method as claimed in claim 48 wherein the density of the encapsulated active substance is between 0.01 and 1 g/cc

52. A method as claimed in claim 1 wherein red blood cell vesicles are loaded with active substance, said method comprising: drawing human blood into a receptacle, washing the red blood cells, spinning the product thus obtained, removing the supernatant liquid and mixing the packed red blood cells thus obtained with an active substance to obtain a cell suspension, freezing and then thawing the suspension to obtain small vesicles of a size of less than five microns which retain the encapsulated active substance upon storage for several days.

53. The loaded cell product obtained by the method of claims 48, 49, 50 and 51.

54. A method as claimed in claim 43 wherein the mixture of active substance and red blood cells is sonicated.

55. A method as claimed in claim 43 wherein the loaded cells are obtained by drawing human blood into a receptacle, washing the blood in the receptacle with buffered saline solution, spinning the solution thus obtained to pack the red blood cells, mixing the packed red blood cells with at least one active substance selected from the group consisting of contrast enhancing dyes, gadodiamide, gold nanoparticle solution, iodine contrast medium, drugs and magnetic particles, inserting the loaded cells into a container in which the cells are subjected to mechanical impact stress whereby the cell walls are ruptured.

56. A method as claimed in claim 55 wherein the size of the loaded vesicles is increased by heating them to approximately 100° C. for one to four minutes whereby the red blood cell vesicles fuse to form larger vesicles.

57. A method as claimed in claim 1 wherein the active substance comprises bacteria and/or viruses, inactivated bacteria and viruses or bacterial and viral components.

58. The process of lysing cells, cell-derived vesicles, or synthetic vesicles loaded with a drug or agent (vesicular carrier) in an animal or human, thus releasing said drug or agent, comprising the steps of:
1. loading said drug or agent in cells, cell-derived vesicles, or synthetic vesicles
2. choosing a membrane of said cells, cell-derived vesicles, or synthetic vesicles that has on its surface an antigen or molecule that can potentially activate the complement or immune system, or linking such an antigen or molecule to said membrane.
3. Administering said vesicular carriers to an animal or human.
4. The said vesicular carrier is targeted to a region in the body either naturally or by design.
5. Allowing the natural immune or complement system of the animal or human to respond resulting in lysis of said vesicular carrier and release of said drug or agent, or applying in an additional step an antibody or agent that binds specifically to said vesicular carrier that activates the immune or complement system resulting in lysis of said vesicular carrier and release of said drug or agent.

59. The process of claim 58 wherein the order of steps 1 and 2 is reversed.

60. The method of claim 1 wherein the effect of the method is enhanced by magnetic localization.

61. The method of claim 60 wherein the magnetic localization is accomplished by the presence of magnetic particles, cooperating with a magnetic field.
